SEQUENCE LISTING (8 sequences)

SEQ ID NO: 1 - protein, 351 aa, Mus musculus
MIIQRVVLNSRPGKNGNPVAENFRVEEFSLPDALNEGQVQVRTLYLSVDPYMRCKMNEDTGTDYLAPWQLAQVADGGGIGVVEESKHQKLTKGDFVTSFYWPWQTKAILDGNGLEKVDPQLVDGHLSYFLGAIGMPGLTSLIGVQEKGHISAGSNQTMVVSGAAGACGSLAGQIGHLLGCSRVVGICGTQEKCLFLTSELGFDAAVNYKTGNVAEQLREACPGGVDVYFDNVGGDISNAVISQMNENSHIILCGQISQYSNDVPYPPPLPPAVEAIRKERNITRERFTVLNYKDKFEPGILQLSQWFKEGKLKVKETMAKGLENMGVAFQSMMTGGNVGKQIVCISEDSSL

SEQ ID NO: 2 - DNA, 1056 bp, Mus musculus
  CDS (1)..(1053)
atgatcatacaaagagtggtattgaattcccgacctgggaaaaatggaaatccagtcgcagagaacttcagggtggaagagttcagtttaccggatgctctcaatgaaggtcaagttcaagtgaggactctttatctctcggtggatccttacatgcgctgtaagatgaacgaggacactggcactgactacttggcaccgtggcagctggcgcaggtggctgatggtggaggaattggagttgtagaggagagcaagcaccagaagttgactaaaggcgattttgtgacttcgttttactggccctggcaaactaaggcaattctagatgggaatggccttgaaaaggtagacccacaacttgtagatggacacctttcatattttcttggggctataggtatgcctggcttgacttccttgattggggtacaggagaaaggccatatatctgctggatctaatcagacaatggttgtcagtggagcagcaggcgcctgtggatctttggctgggcagattggccacctgcttggctgttccagagtggtgggaatttgtggaacgcaggagaaatgtctctttttgacctcagagctggggtttgatgctgcagttaattacaaaacagggaatgtggcagagcagctgcgagaagcgtgcccgggcggagtggatgtctactttgacaatgttggaggtgacatcagcaacgcggtgataagtcagatgaatgagaacagccacatcatcctgtgtggtcagatttctcagtacagtaacgatgtgccctaccctcctccactgccccctgcagtagaagccatccggaaggaacgaaacatcacaagagagagatttacggtattaaattataaagataaatttgagcctggaattctacagctgagtcagtggtttaaagaaggaaagctaaaggtcaaggagaccatggcaaagggcttggaaaacatgggagttgcattccagtccatgatgacagggggcaacgtagggaaacagatcgtctgcatttcagaagattcttctctgtag

SEQ ID NO: 3 - DNA, 20 bp, Artificial Sequence
  Description of Artificial Sequence Synthetic primer
cggtatagcttgggacgcta

SEQ ID NO: 4 - DNA, 22 bp, Artificial Sequence
  Description of Artificial Sequence Synthetic primer
tgcatgttaagaatctttgtgg

SEQ ID NO: 5 - DNA, 26 bp, Artificial Sequence
  Description of Artificial Sequence Synthetic primer
aactgaagcttcaagtgatgatcata

SEQ ID NO: 6 - DNA, 22 bp, Artificial Sequence
  Description of Artificial Sequence Synthetic primer
agctctcccatatggtcgacct

SEQ ID NO: 7 - RNA, 19 nt, Artificial Sequence
  Description of Artificial Sequence Synthetic oligonucleotide
gaguucaguuuaccggaug

SEQ ID NO: 8 - RNA, 19 nt, Artificial Sequence
  Description of Artificial Sequence Synthetic oligonucleotide
guucaagugaggacucuuu

(19) United States
(12) Patent Application Publication
(10) Pub. No.: US 2009/0124688 A1
(43) Pub. Date: May 14, 2009

(54) Prostaglandin reductase inhibitors

(76) Inventors: Rong-Hwa Lin, Los Altos, CA (US); Leewen Lin, Taipei (TW); Shih-Yao Lin, Taipei (TW); Shu-Hua Lee, Taipei (TW)

Correspondence Address: OCCHIUTI ROHLICEK & TSAO, LLP, 10 FAWCETT STREET, CAMBRIDGE, MA, 02138, US

(21) Appl. No.: 11/650,868

(22) Filed: Jan. 8, 2007

Related U.S. Application Data
(60) Provisional application No. 60/756,734, filed on Jan. 6, 2006.

(51) Int. Cl. A61K 31/215; C12N 9/99; A61K 31/19

(52) U.S. Cl. 514/530; 435/184; 514/573

(57) ABSTRACT

A method of inhibiting 15-keto prostaglandin-Δ¹³-reductase 2 by contacting 15-keto prostaglandin-Δ¹³-reductase 2 with an aryl compound of Formula (I), (II), (III), or (IV) shown herein. Also disclosed are methods of treating peroxisome proliferators-activated receptor related diseases and lowering blood glucose levels by administering to a subject in need thereof an effective amount of such an aryl compound.

### CROSS REFERENCE TO RELATED APPLICATIONS

This application claims priority to U.S. Provisional Application Ser. No. 60/756,734, filed on Jan. 6, 2006, the contents of which are incorporated herein by reference in their entirety.

### BACKGROUND

Peroxisome proliferator-activated receptors (PPARs) belong to a family of nuclear receptors that regulate lipid and glucose metabolism. Three mammalian PPARs have been identified, i.e., PPAR-α, PPAR-γ, and PPAR-δ. Upon activation by either dietary fatty acids, PPARs trigger a cascade of transcriptional events leading to altered lipid and glucose metabolism. For example, activated PPAR-γ promotes glucose uptake and lowers blood glucose levels.

Given their roles in lipid and glucose metabolism, PPARs are promising therapeutic targets of diseases, e.g., type II diabetes, obesity, dyslipidemia, coronary heart disease, inflammatory disease, and cancer. For example, Avandia, a synthetic PPAR-γ agonist, has been used to treat type II diabetes and Fibrate, another synthetic PPAR-α agonist, has been used to treat dyslipidemia. See Lehmann, et al., J Biol Chem, (1995) 270:12953-12956; Fruchart, et al., Curr. Opin. Lipdol. (1999) 10:245-257. However, most PPARs therapeutics have limited efficacy and significant side effects.

There is a need to develop more effective drugs for controlling lipid and glucose metabolism via modulatiing PPARs activity.

### SUMMARY

The present invention is based on surprising findings that modulators of 15-keto prostaglandin-Δ¹³-reductase 2 (15-keto PGR-2) controlled the activity of PPARs and that a number of aryl compounds unexpectedly inhibited activity of 15-keto PGR-2. 15-keto PGR-2 is an enzyme of the 15-keto prostaglandin-Δ¹³-reductase family. It reduces 15-keto prostaglandin, but not leukotriene B4. See, e.g., U.S. application Ser. No. 11/147,711.

In one aspect, this invention features a method of inhibiting 1 5-keto PGR-2 by contacting this enzyme with one or more aryl compounds.

In one embodiment, the aryl compounds mentioned above have formula (I):

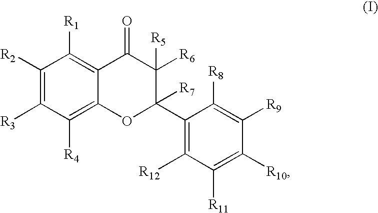

wherein each of R₁, R₂, R₃, R₄, R₅, R₆, R₇, R₈, R₉, R₁₀ , R₁₁, and R₁₂, independently, is H, OH, C₁-C₁₀ alkoxy, C₁-C₁₀ alkyl, C₃-C₂₀ cycloalkyl, C₃-C₂₀ heterocycloalkyl, aryl, or heteroaryl; in which R is H, C₁-C₁₀ alkyl, C₃-C₂₀ cycloalkyl, C₃-C₂₀ heterocycloalkyl, aryl, or heteroaryl; or R₆ and R₇, taken together, represent a bond.

In another embodiment, the aryl compounds mentioned above have formula (II):

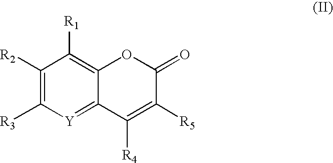

in which Y is N or CR₆; each of R₁, R₂, R₃, and R₆, independently, is H, halo, OR, C₁-C₁₀ alkyl, carboxy, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₁ and R₂, R₂ and R₃, or R₃ and R₆, together with the two carbon atoms to which they are attached, form C₃-C₂₀ cycloalkyl, C₃-C₂₀ heterocycloalkyl, aryl, or heteroaryl; R₄ is H, halo, OR, C₁-C₁₀ alkyl, carboxy, C₃-C₂₀ cycloalkyl, C₃-C₂₀ heterocycloalkyl, aryl, or heteroaryl, in which R is defined above; and R₅ is H, halo, OR, C₁-C₁₀ alkyl, carboxy, C₃-C₂₀ cycloalkyl, C₃-C₂₀ heterocycloalkyl, aryl, or heteroaryl, in which R is defined above;

### or R₅ is

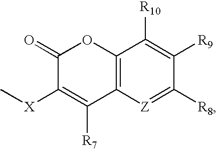

in which X is O, S, NR′, C(O), or CR′R″; each R′ and R″, independently, being H, OH, C₁-C₁₀ alkoxyl, halo, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R′ and R″, together with the carbon atom to which they are attached, being C₃-C₂₀ cycloalkyl, C₃-C₂₀ heterocycloalkyl, aryl, or heteroaryl; Y is N or CR₁₁,; R₇ is H, OH, C₁-C₁₀ alkoxyl, halo, C₁-C₁₀ alkyl, C₃-C₂₀ cycloalkyl, C₃-C₂₀ heterocycloalkyl, aryl, or heteroaryl; each of R₈, R₉, R₁₀, and R₁₁, independently, being H, OH, C₁-C₁₀ alkoxyl, halo, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₈ and R₉, R₉ and R₁₀, or R₈ and R₁₁, together with the two carbon atoms to which they are attached, form C₃-C₂₀ cycloalkyl, C₃-C₂₀ heterocycloalkyl, aryl, or heteroaryl.

On subset of the above compounds features that R₅ is

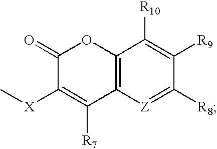

Y is CR₆; Z is CR₁₁; X is C(O) or CHR′, R′ being H, aryl or heteroaryl; each of R₁, R₂, R₃, R₄, R₅, R₇, R₈, R₉, R₁₀, and R₁₁, is H, OH, OMe, or halo. Another subset features that Y is CR₆; each of R₁, R₂, R₃, R₄, and R₆ is H, OH, OMe, or Me; and R₅ is H or alkyl optionally substituted with carboxy, carbonyl, alkyloxycarbonyl, aryloxycarbonyl, or heteroaryl.

In still another embodiment, the aryl compounds mentioned above have formula (III):

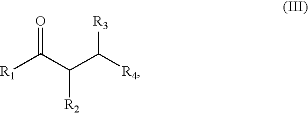

wherein each of R₁ and R₄, independently, is H, OR, SR, NRR′, C₁-C₁₀ alkyl, C₃-C₂₀ cycloalkyl, C₃-C₂₀ heterocycloalkyl, aryl, or heteroaryl, in which each of R and R′, independently, is H, C₁-C₁₀ alkyl, C₃-C₂₀ cycloalkyl, C₃-C₂₀ heterocycloalkyl, aryl, or heteroaryl; and each of R₂ and R₃, independently, is H, OR, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₂ and R₃, taken together, represent a single bond or double bond.

One subset of the above compounds features that each of R₁ and R4, independently, is aryl (e.g., phenyl, optionally substituted with H, OR, halo, nitro, cyano, C₁-C₁₀ alkyl, C₃-C₂₀ cycloalkyl, C₃-C₂₀ heterocycloalkyl, aryl, or heteroaryl, R being H, C₁-C₁₀ alkyl, C₃-C₂₀ cycloalkyl, C₃-C₂₀ heterocycloalkyl, aryl, or heteroaryl); or heteroaryl (e.g., furyl); each of R₂ and R₃, taken together, represent a single bond; and each of R₆ and R₇ is H.

Yet, in another embodiment, the aryl compounds mentioned above have formula (IV):

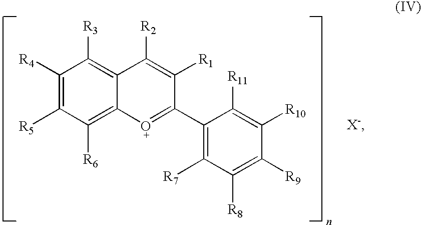

wherein each of R₁, R₂, R₃, R₄, R₅, R₆, R₇, R₈, R₉, R₁₀, and R₁₁, independently, is H, C₁-C₁₀ alkoxy, halo, C₁-C₁₀ alkyl, C₃-C₂₀ cycloalkyl, C₃-C₂₀ heterocycloalkyl, aryl, or heteroaryl; in which R is H, C₁-C₁₀ alkyl, C₃-C₂₀ cycloalkyl, C₃-C₂₀ heterocycloalkyl, aryl, or heteroaryl; X is an anion; and n is the absolute value of the charge of X.

Shown below are exemplary compounds that can be used as 15-keto PGR-2 inhibitors:

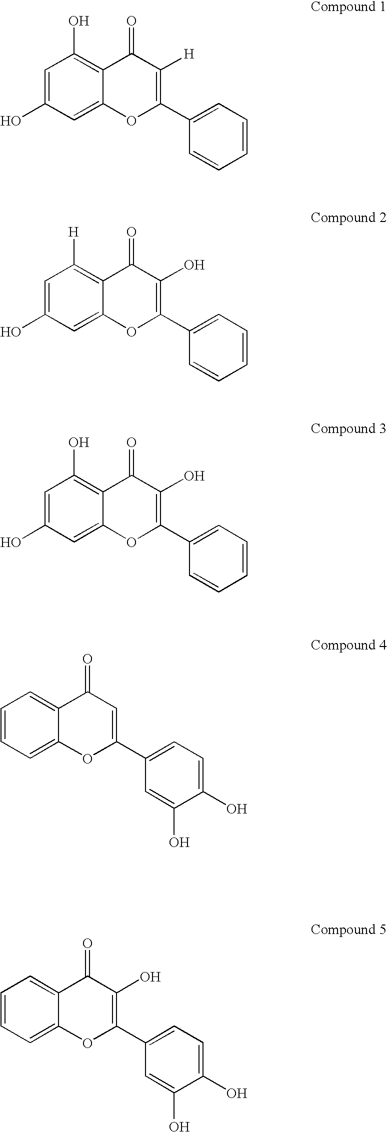

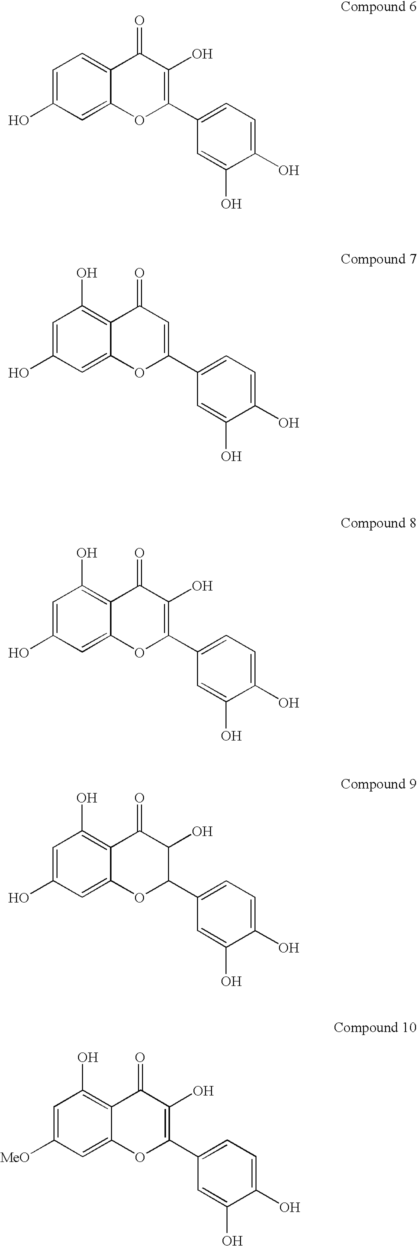

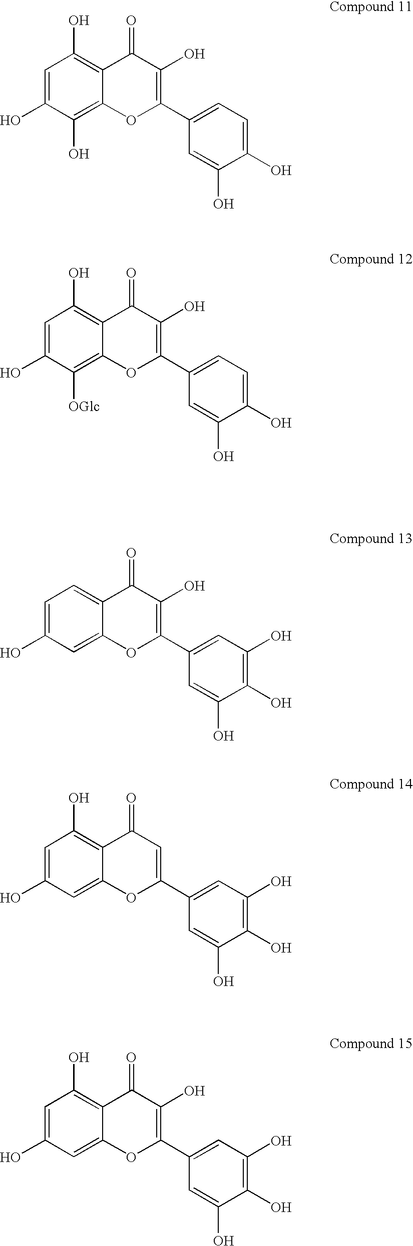

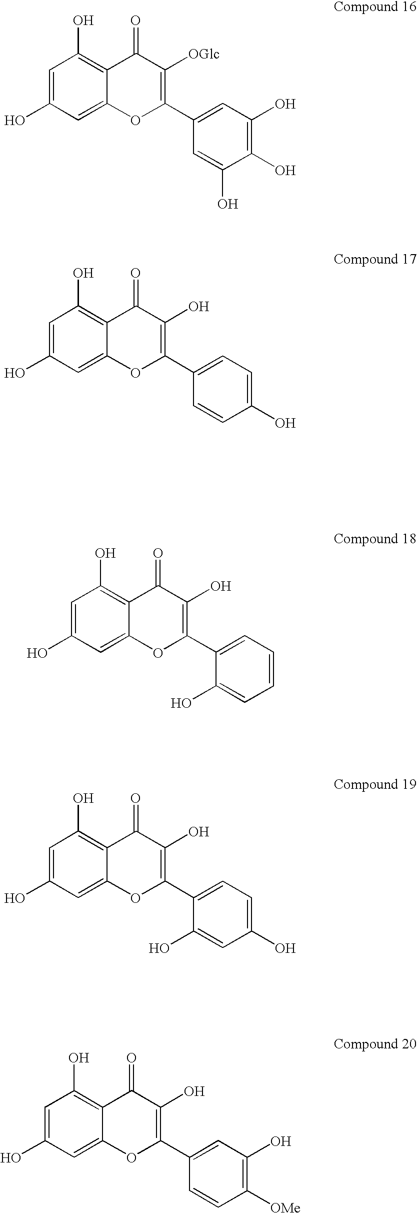

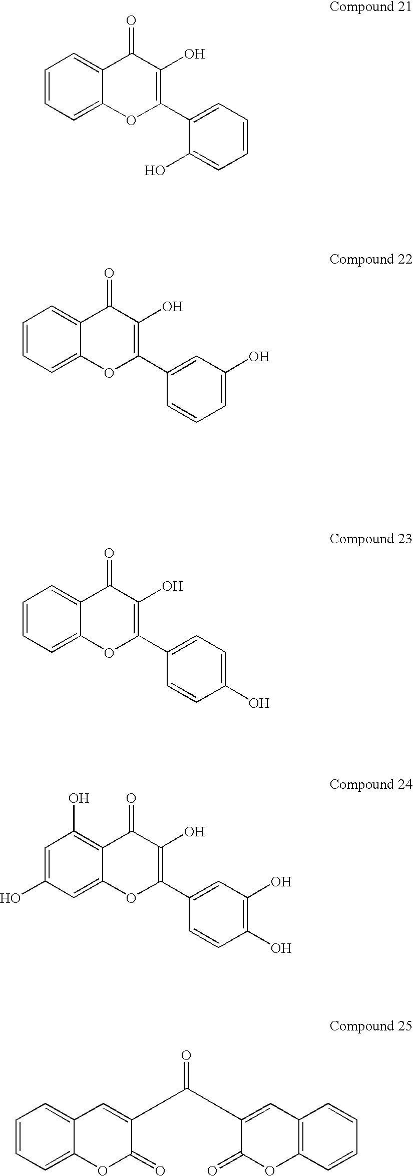

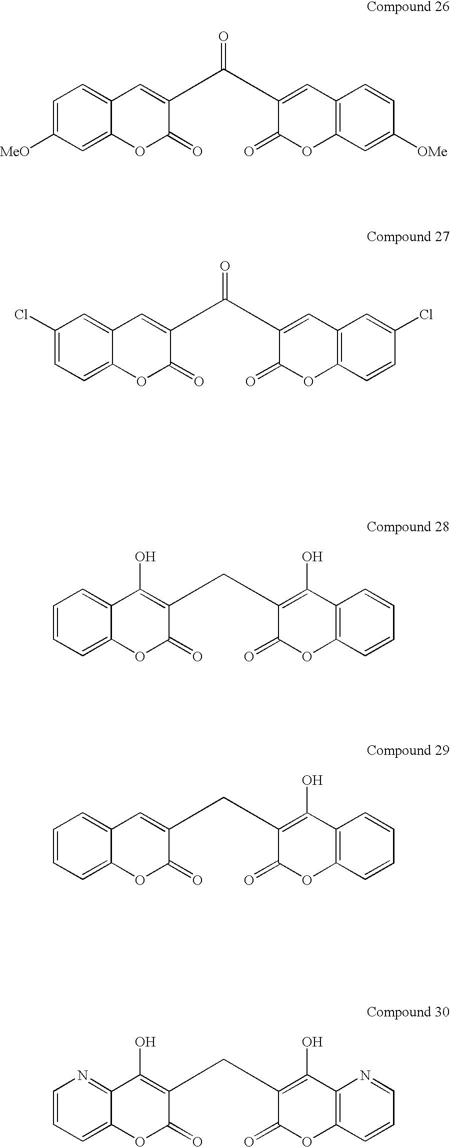

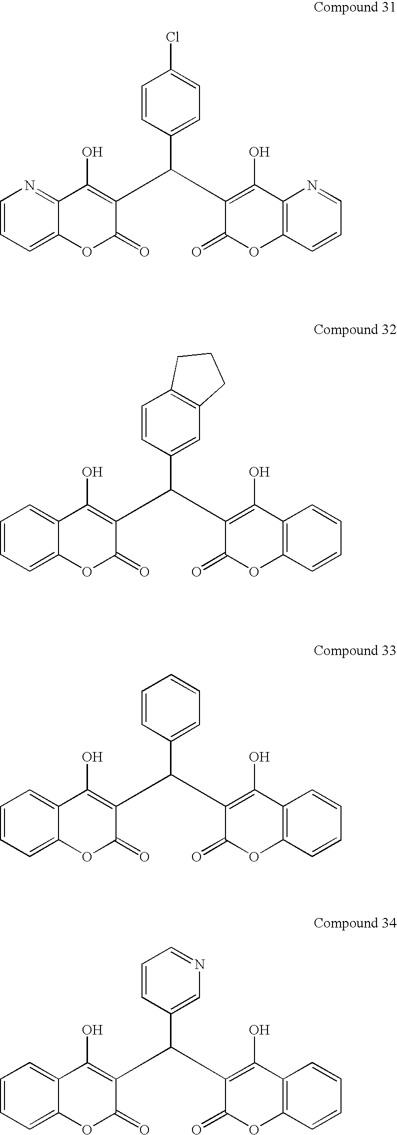

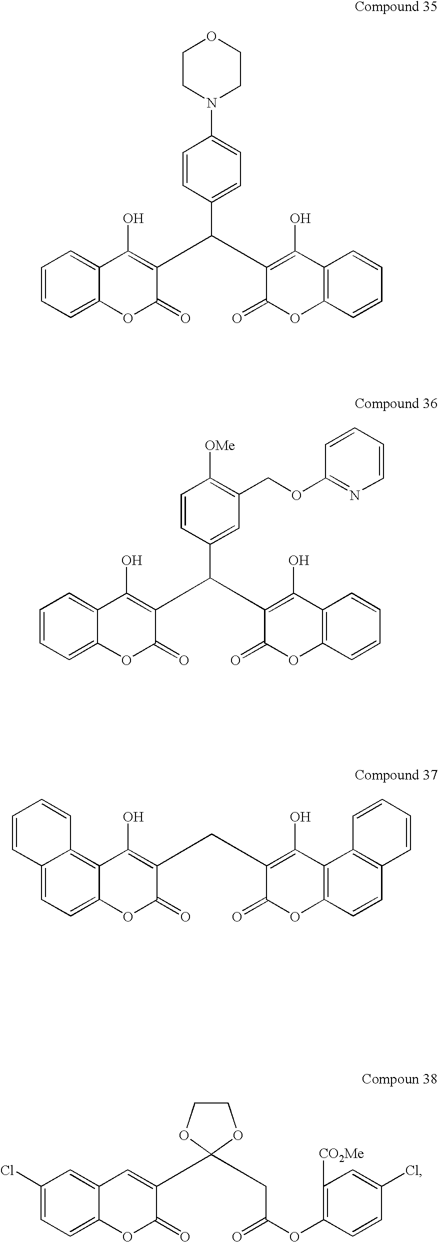

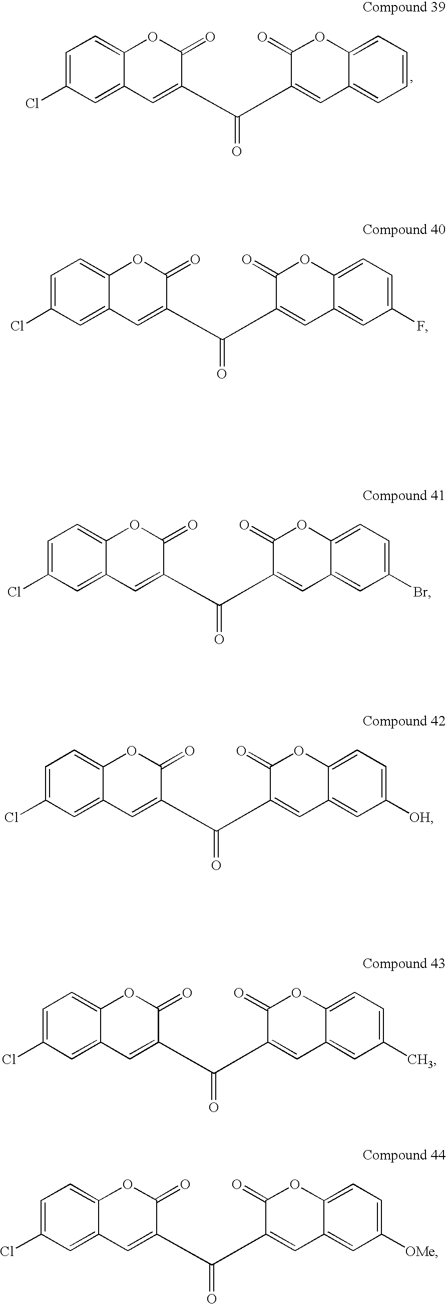

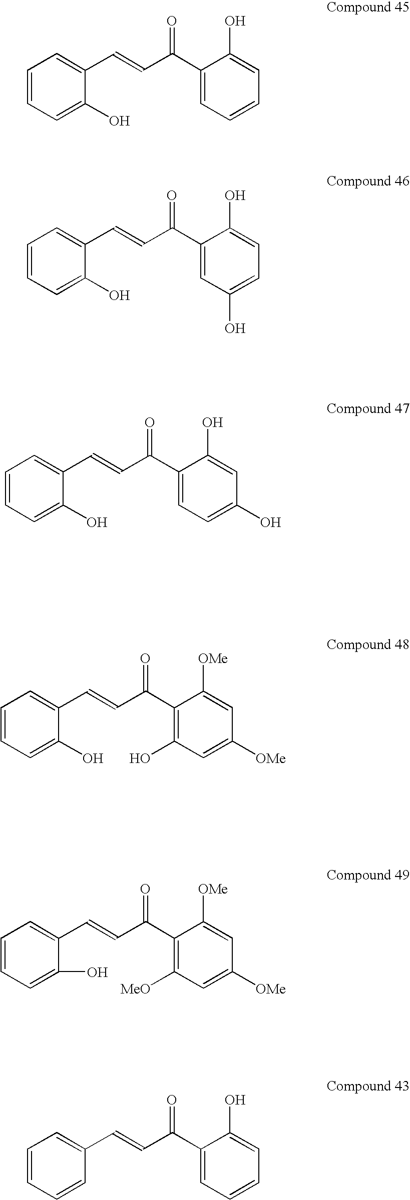

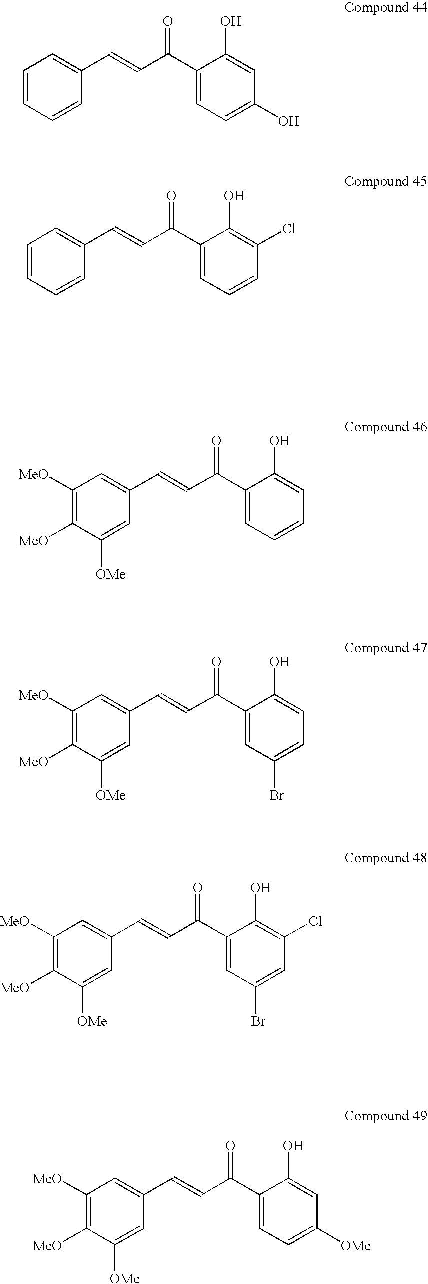

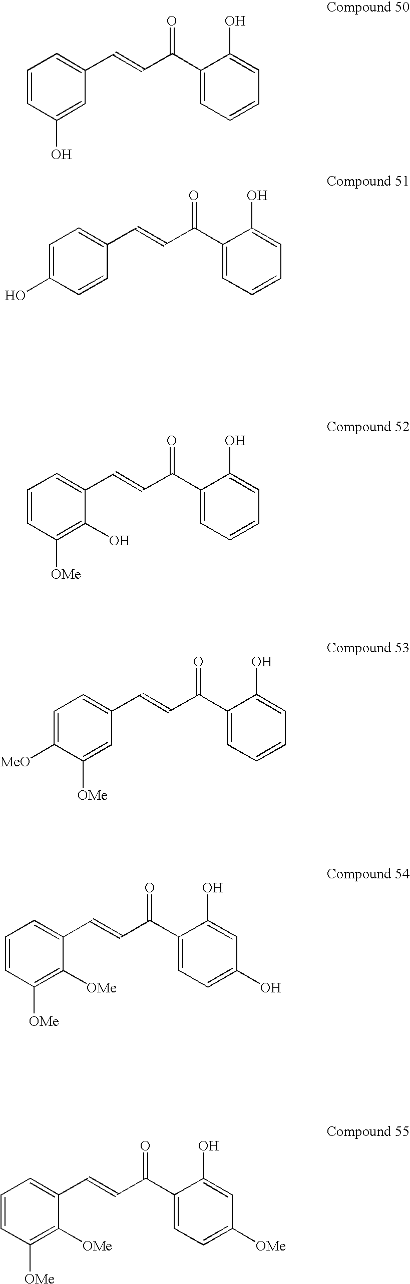

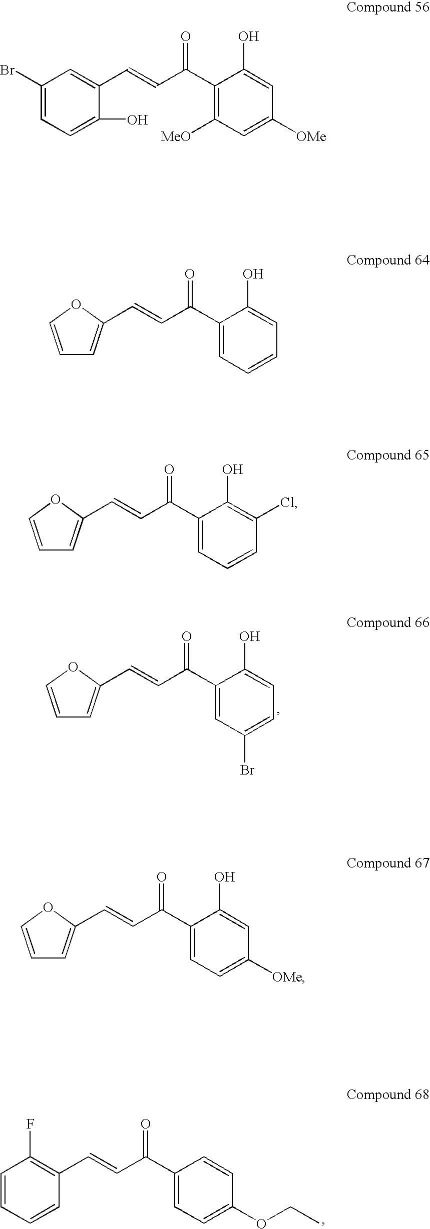

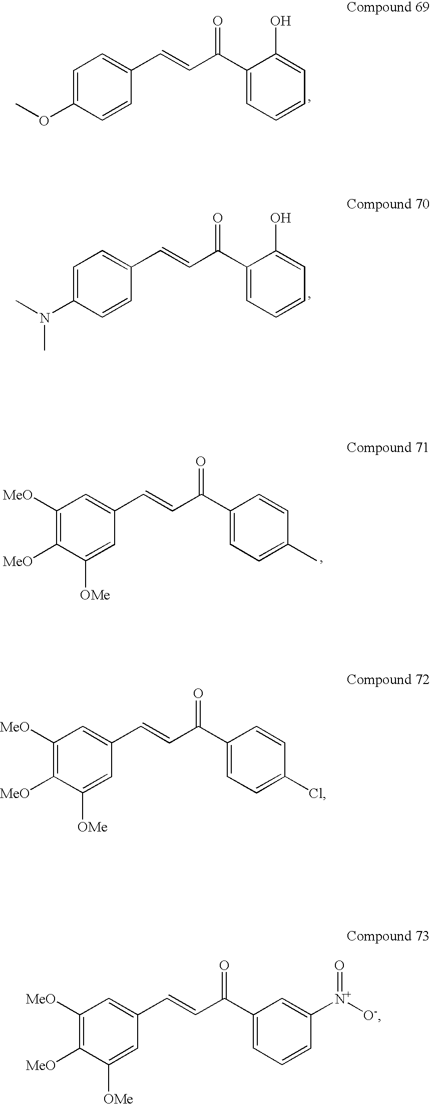

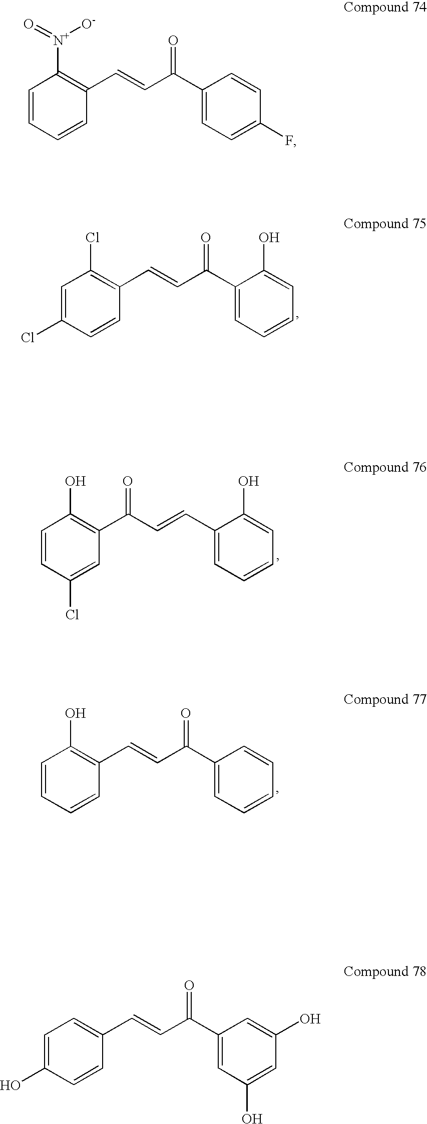

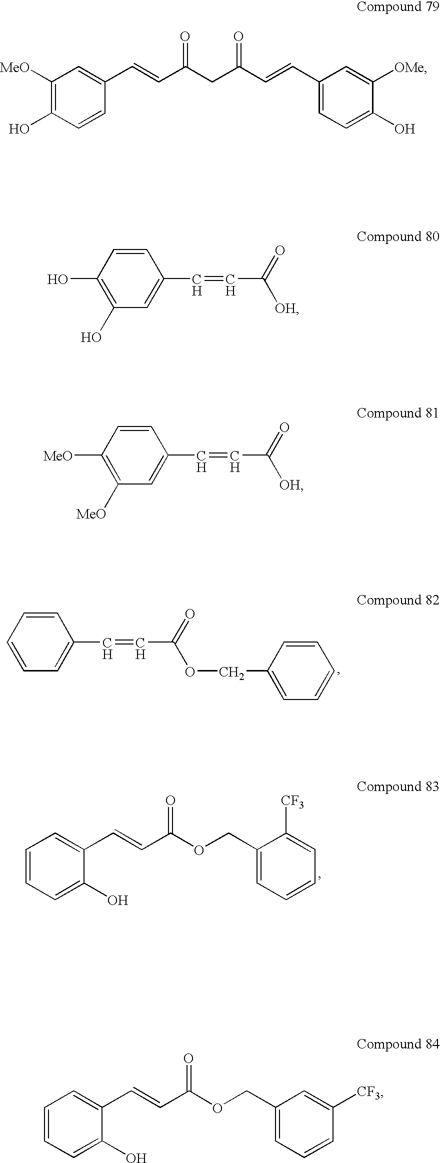

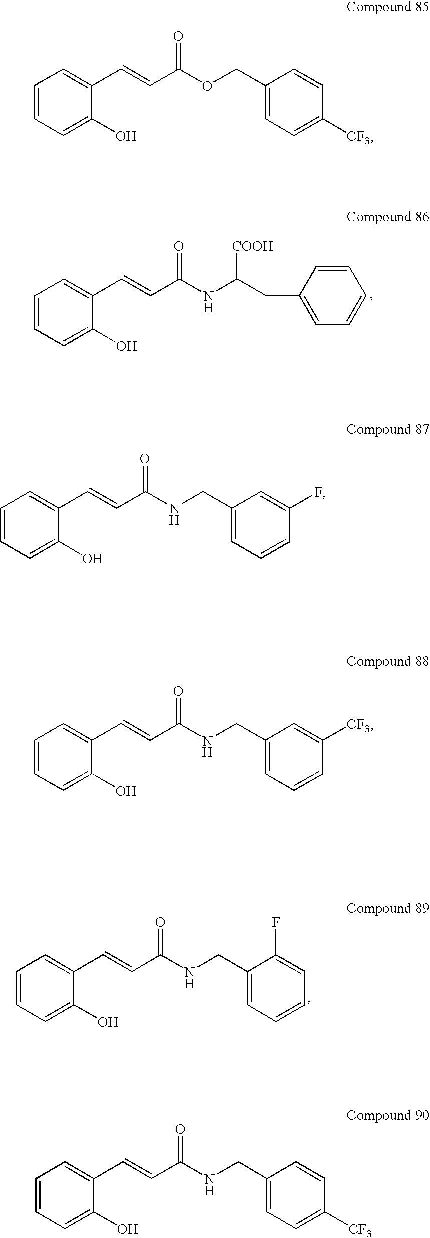

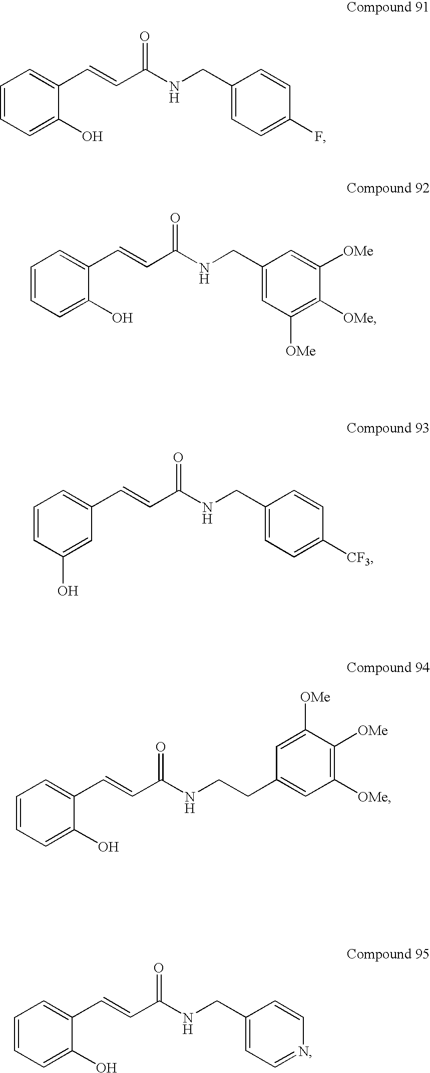

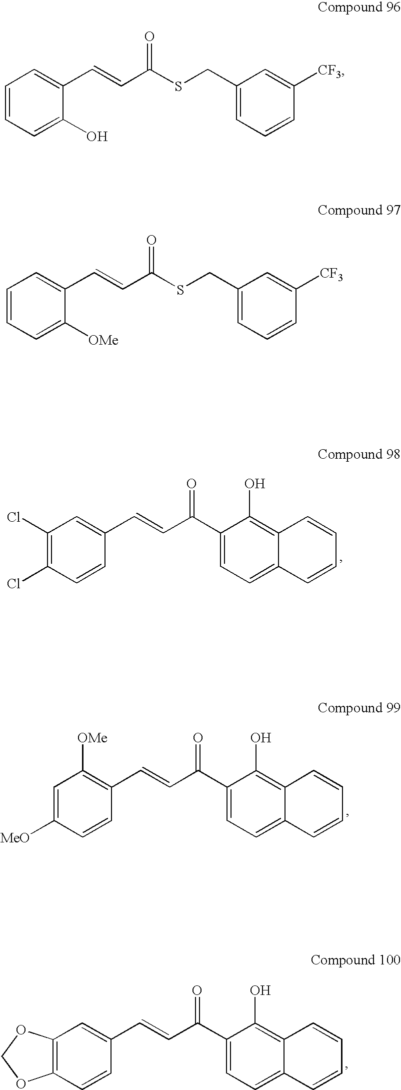

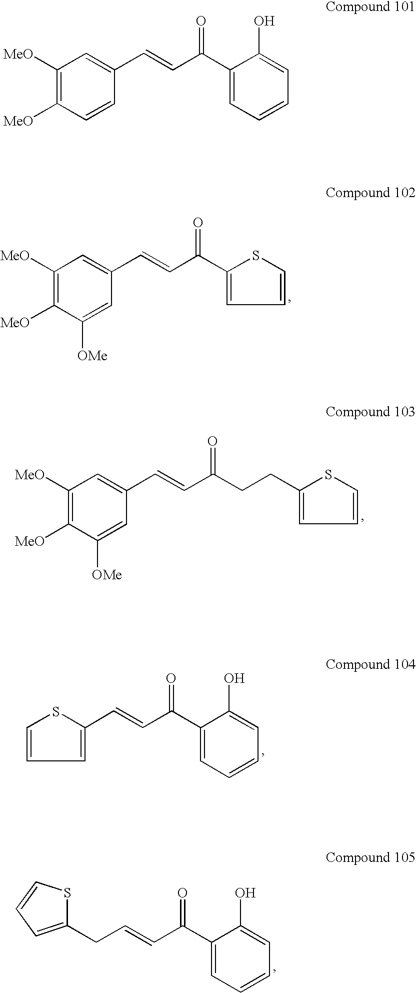

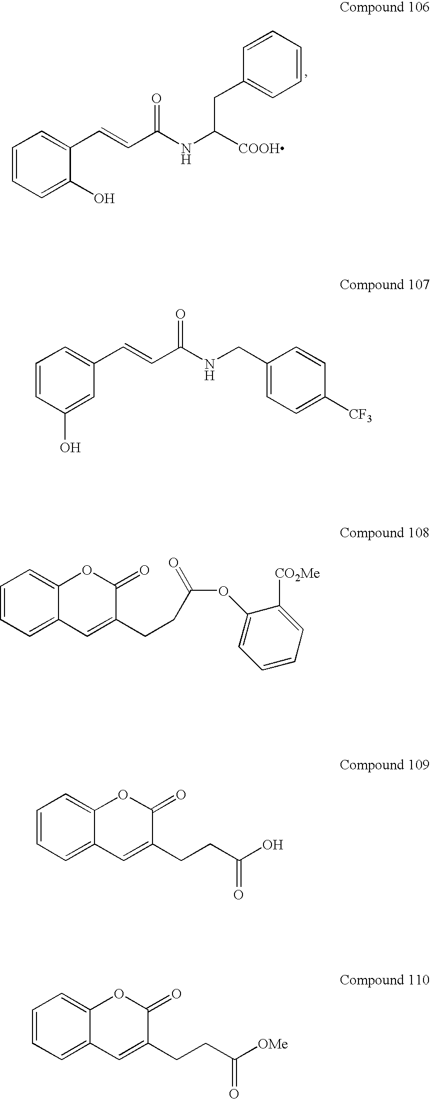

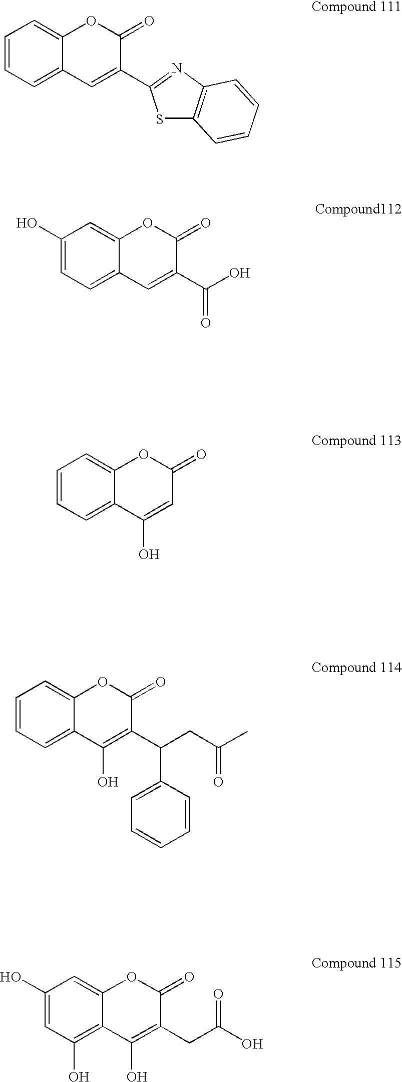

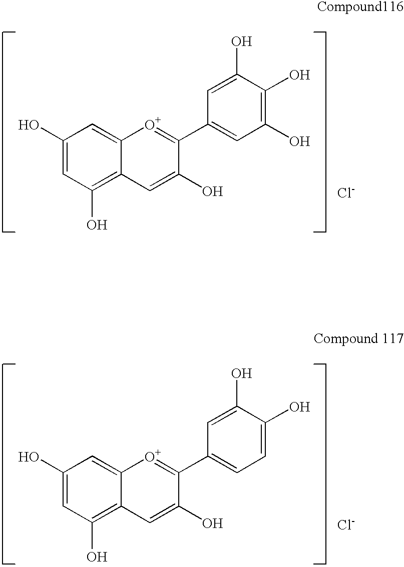

The term “alkyl” herein refers to a straight or branched hydrocarbon, containing 1-10 carbon atoms. Examples of alkyl groups include, but are not limited to, methyl, ethyl, n-propyl, i-propyl, n-butyl, i-butyl, and t-butyl. The term “alkoxy” refers to an —O-alkyl. The term “alkoxyalkyl” refers to an alkyl group substituted with one or more, groups. The term “haloalkyl” refers to an alkyl group substituted with one or more halo groups. The term “hydroxyalkyl” refers to an alkyl group substituted with one or more hydroxy groups.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl. The term “aryloxy” refers to an —O-aryl. The term “aralkyl” refers to an alkyl group substituted with an aryl group.

The term “cycloalkyl” refers to a saturated and partially unsaturated cyclic hydrocarbon group having 3 to 12 carbons. Examples of cycloalkyl groups include, but are not limited to, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl. The term “heteroaralkyl” refers to an alkyl group substituted with a heteroaryl group.

The term “heterocycloalkyl” refers to a nonaromatic 5-8 membered monocyclic, 8-12 membered bicyclic, or 11-14 membered tricyclic ring system having one or more heteroatoms (such as O, N, or S). Examples of heterocycloalkyl groups include, but are not limited to, piperazinyl, pyrrolidinyl, dioxanyl, morpholinyl, and tetrahydrofuranyl. Heterocycloalkyl can be a saccharide ring, e.g., glucosyl.

Alkyl, cycloalkyl, heterocycloalkyl, aryl, heteroaryl, aralkyl, heteroaralkyl, alkoxy, and aryloxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cycloalkyl, heterocycloalkyl, in which alkyl, alkenyl, alkynyl, alkyloxy, aryl, heteroaryl cycloalkyl, and heterocycloalkyl may further substituted.

The term “anion” refers to a negatively charged ion. Examples of an anion include, but are not limited to, Cl⁻, Br⁻, I⁻, SO₄²⁻, PO₄³⁻, CIO₄⁻, CH₃CO₂⁻, and CF₃CO₂⁻.

Modulators of 15-keto PGR-2 (i.e., substrates and inhibitors of the enzyme) can control PPARs activity. These substrates and inhibitors are useful for treating PPAR related diseases. Thus, in another aspect, this invention also features a method of treating a PPARs related disease such as type II diabetes, obesity, dyslipidemia, coronary heart disease, inflammatory disease, and cancer. The method includes administering to a subject an effective amount of a 15-keto PGR-2 modulator. A 15-keto PGR-2 modulator refers to a molecule or a complex of molecules that affects activity or expression of this enzyme. A modulator can be a 15-keto prostaglandin, e.g., 15-keto PGE₂, 15-keto PGE₁, 15-keto PGF_{2α}, 15-keto PGF_{1α}, 15-keto fluprostenol isopropyl ester, or 15-keto fluprostenol. It can also be an inhibitor that suppresses either activity or expression of 15-keto prostaglandin-α¹³-reductase 2. Examples of such an inhibitor include the aryl compounds of any of formulas (I), (II), (III), and (IV).

Further, this invention features a method of lowering blood glucose levels by administering to a subject an effective amount of a 15-keto PGR-2 modulator.

Also within the scope of this invention is a composition containing a 15-keto PGR-2 modulator (e.g., a compound of any of formulas (I), (II), (III), and (IV)) and a pharmaceutically acceptable carrier for use in treating PPAR related diseases or lowering blood glucose levels, as well as the use of such a composition for the manufacture of a medicament for treating PPAR related diseases or lowering blood glucose levels.

The details of one or more embodiments of the invention are set forth in the description below. Other features, objects, and advantages of the invention will be apparent from the description and from the claims.

### DETAILED DESCRIPTION

Shown below is the amino acid sequence of 15-keto PGR-2 (SEQ ID NO:1), as well as its encoding nucleotide sequence (i.e., SEQ ID NO:2).

| | |
|---|---|
| 1 - ATGATCATACAAAGAGTGGTATTGAATTCCCGACCTGGGAAAAATGGAAATCCAGTCGCA - 60 | (SEQ ID NO:2) |
| - M I I Q R V V L N S R P G K N G N P V A | (SEQ ID NO:1) |
| 61 - GAGAACTTCAGGGTGGAAGAGTTCAGTTTACCGGATGCTCTCAATGAAGGTCAAGTTCAA - 120 | |
| - E N F R V E E F S L P D A L N E G Q V Q | |
| 121 - GTGAGGACTCTTTATCTCTCGGTGGATCCTTACATGCGCTGTAAGATGAACGAGGACACT - 180 | |
| - V R T L Y L S V D P Y M R C K M N E D T | |
| 181 - GGCACTGACTACTTGGCACCGTGGCAGCTGGCGCAGGTGGCTGATGGTGGAGGAATTGGA - 240 | |
| - G T D Y L A P W Q L A Q V A D G G G I G | |
| 241 - GTTGTAGAGGAGAGCAAGCACCAGAAGTTGACTAAAGGCGATTTTGTGACTTCGTTTTAC - 300 | |
| - V V E E S K H Q K L T K G D F V T S F Y | |
| 301 - TGGCCCTGGCAAACTAAGGCAATTCTAGATGGGAATGGCCTTGAAAAGGTAGACCCACAA - 360 | |
| - W P W Q T K A I L D G N G L E K V D P Q | |
| 361 - CTTGTAGATGGACACCTTTCATATTTTCTTGGGGCTATAGGTATGCCTGGCTTGACTTCC - 420 | |
| - L V D G H L S Y F L G A I G M P G L T S | |
| 421 - TTGATTGGGGTACAGGAGAAAGGCCATATATCTGCTGGATCTAATCAGACAATGGTTGTC - 480 | |
| - L I G V Q E K G H I S A G S N Q T M V V | |
| 481 - AGTGGAGCAGCAGGCGCCTGTGGATCTTTGGCTGGGCAGATTGGCCACCTGCTTGGCTGT - 540 | |
| - S G A A G A C G S L A G Q I G H L L G C | |
| 541 - TCCAGAGTGGTGGGAATTTGTGGAACGCAGGAGAAATGTCTCTTTTTGACCTCAGAGCTG - 600 | |
| - S R V V G I C G T Q E K C L F L T S E L | |
| 601 - GGGTTTGATGCTGCAGTTAATTACAAAACAGGGAATGTGGCAGAGCAGCTGCGAGAAGCG - 660 | |
| - G F D A A V N Y K T G N V A E Q L R E A | |
| 661 - TGCCCGGGCGGAGTGGATGTCTACTTTGACAATGTTGGAGGTGACATCAGCAACGCGGTG - 720 | |
| - C P G G V D V Y F D N V G G D I S N A V | |
| 721 - ATAAGTCAGATGAATGAGAACAGCCACATCATCCTGTGTGGTCAGATTTCTCAGTACAGT - 780 | |
| - I S Q M N E N S H I I L C G Q I S Q Y S | |
| 781 - AACGATGTGCCCTACCCTCCTCCACTGCCCCCTGCAGTAGAAGCCATCCGGAAGGAACGA - 840 | |
| - N D V P Y P P P L P P A V E A I R K E R | |
| 841 - AACATCACAAGAGAGAGATTTACGGTATTAAATTATAAAGATAAATTTGAGCCTGGAATT - 900 | |
| - N I T R E R F T V L N Y K D K F E P G I | |
| 901 - CTACAGCTGAGTCAGTGGTTTAAAGAAGGAAAGCTAAAGGTCAAGGAGACCATGGCAAAG - 960 | |
| - L Q L S Q W F K E G K L K V K E T M A K | |
| 961 - GGCTTGGAAAACATGGGAGTTGCATTCCAGTCCATGATGACAGGGGGCAACGTAGGGAAA - 1020 | |
| - G L E N M G V A F Q S M M T G G N V G K | |
| 1021 - CAGATCGTCTGCATTTCAGAAGATTCTTCTCTGTAG - 1056 | |
| - Q I V C I S E D S S L * | |

This invention relates to a method of inhibiting 15-keto PGR-2. The method includes contacting this enzyme with an effective amount of a compound of formula (I), (II), (III), or (IV) described above. Inhibition refers to suppression of either activity or expression of 15-keto PGR-2.

15-keto PGR-2 activity refers to the enzymatic conversion of 15-keto prostaglandin to 13,14-dihydro-15-keto prostaglandin. The specific activity is determined as follows: 5 μg of recombinant mouse or human prostaglandin-Δ¹³-reductase 2/zinc binding alcohol dehydrogenase I (PGR2/ZADH1) protein preparation to be assayed is incubated at 37° C. in 50 μl of reaction buffer containing 0.1 M Tris-HCl (pH 7.4), 0.5 mM NADPH, and 0.57 mM 15-keto PGE₂. The reaction solution is kept in the dark for 2 hours at 37° C. and then mixed with 40 μl of a color development buffer containing 790 μM indonitrotetraolium chloride, 60 μM phenazene methosulfate, and 1% Tween 20 to oxidize any unreacted NADPH. After 10 min in the dark, 140 μl of a color development reagent containing 50 mM potassium hydrogen phthalate, pH 3.0, and 1% Tween 20 is added. Absorbance at 490 nm is measured using an ELISA plate reader. A standard curve is generated using reaction buffers containing serially diluted amounts of NADPH. A specific activity of at least 90 nmole/min/mg protein indicates that the polypeptide has 15-keto prostaglandin-Δ¹³-reductase 2 activity.

The compounds of formula (I), (II), (III), and (IV) can be used to inhibit 15-keto prostaglandin-Δ¹³-reductase 2 activity. Some of them are available from commercial sources. They can also be synthesized by conventional methods. Shown below are three schemes illustrating synthetic routes to some of these compounds.

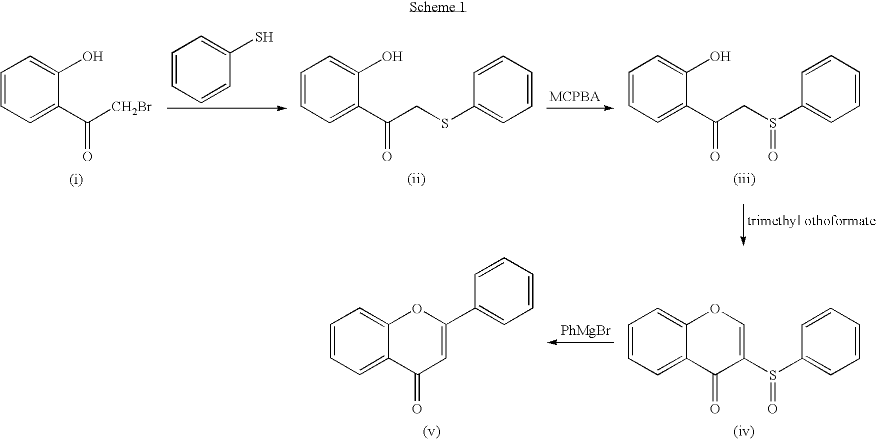

As shown above, 2-bromo-1-(2-hydroxyphenyl)ethanone (i) is reacted with benzenethiol to a 2-(phenylthio)ethanone compound (ii), which is subsequently oxidized to 2-(phenylsulfinyl)ethanone (iii) by an oxidizing agent, e.g., meta-Chloroperbenzoic acid (MCPBA). Compound (iii) is then reacted with trimethylothoformate to form 3-(phenylsulfinyl)-4H-chromen-4-one (iv), which can be further transformed to 2-phenyl-4H-chromen-4-one (v), a compound of formula (I).

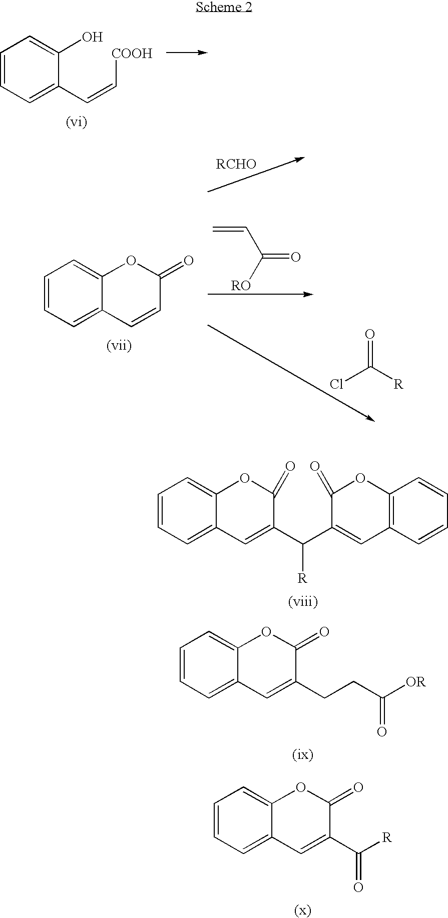

Cyclization of 3-(2-hydroxyphenyl)acrylic acid (vi) affords 2H-chromen-2-one (vii), a compound of formula (II). This compound can be easily transformed to other compounds of formula (II), e.g., compounds (viii), (ix), and (x) as shown above.

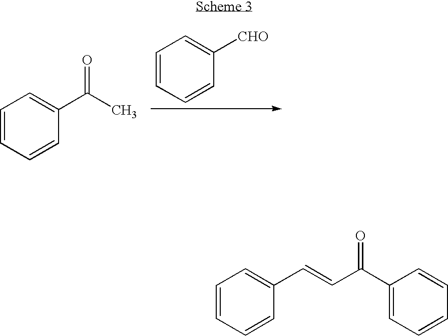

Scheme 3 demonstrates an aldol condensation to form a α,β unsaturated keton compound of formula (III). Hydrogentation of the double bond affords saturated keton compound of formula (III).

Synthetic chemistry transformations useful in synthesizing applicable compounds are described, for example, in R. Larock, Comprehensive Organic Transformations, VCH Publishers (1989); T. W. Greene and P. G. M. Wuts, i Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

This invention also relates to a method of treating PPAR related diseases by modulating 15-keto PGR-2 activity or expression. The term “treating” refers to administering one or more of the above-described 15-keto PGR-2 modulators, i.e., 15-keto PGR-2 substrates and inhibitors, to a subject who has a PPAR related disease, a symptom of such a disease, or a predisposition toward such a disease, with the purpose to confer a therapeutic effect, e.g., to cure, relieve, alter, affect, ameliorate, or prevent the PPAR related disease, the symptom of it, or the predisposition toward it. “An effective amount” refers to the amount that is required to confer a therapeutic effect on a treated subject. Examples of PPAR related diseases (or disorders or conditions) include, but are not limited to, type II diabetes, hyperglycemia, low glucose tolerance, Syndrome X, insulin resistance, obesity, lipid disorders, dyslipidemia, hyperlipidemia, hypertriglyceridemia, hypercholesterolemia, low HDL levels, high LDL levels, atherosclerosis (and its sequelae such as angina, claudication, heart attack, or stroke), vascular stenosis, irritable bowel syndrome, inflammatory diseases (e.g., inflammatory bowel disease, rheumatoid arthritis, Crohn's disease, ulcerative colitis, osteoarthritis, multiple sclerosis, asthma, vasculitis, ischemia/reperfusion injury, frostbite, or adult respiratory distress syndrome), pancreatitis, neurodegenerative disease, retinopathy, neoplastic conditions, cancers (e.g., prostate, gastric, breast, bladder, lung, or colon cancer, or adipose cell cancer such as liposarcoma), angiogenesis, Alzheimer's disease, skin disorders (e.g., acne, psoriasis, dermatitis, eczema, or keratosis), high blood pressure, ovarian hyperandrogenism, osteoporosis, and osteopenia.

To treat a PPAR related disease, a pharmaceutical composition containing a PGR-2 modulator and a pharmaceutically acceptable carrier can be administered to a subject in need thereof. It can be administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The pharmaceutical composition can be a solution or suspension in a non-toxic acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

The dosage required depends on the choice of the route of administration; the nature of the formulation; the nature of the subject's illness; the subject's size, weight, surface area, age, and sex; other drugs being administered; and the judgment of the attending physician. Suitable dosages may be in the range of 0.01-100.0 mg/kg. Wide variations in the needed dosage are to be expected in view of the variety of compositions available and the different efficiencies of various routes of administration. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

The above-described pharmaceutical composition can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of the above-described pharmaceutical composition can be evaluated both in vitro and in vivo. Briefly, the pharmaceutical composition can be tested for its ability to inhibit PGR-2 activity or expression in vitro. For in vivo studies, the pharmaceutical composition can be injected into an animal (e.g., a mouse model) having a PPAR disease or high glucose levels and its therapeutic effects are then accessed. Based on the results, an appropriate dosage range and administration route can be determined.

The invention further features a method of inhibiting PGR-2 activity or expression using chemical compounds. The compounds can be designed, e.g., using computer modeling programs, according to the three-dimensional conformation of the polypeptide, and synthesized using methods known in the art. It can also be identified by library screening, or obtained using any of the numerous approaches in combinatorial library methods known in the art. Suitable libraries include: peptide libraries, peptoid libraries (libraries of molecules having the functionalities of peptides, but with a novel, non-peptide backbone that is resistant to enzymatic degradation), spatially addressable parallel solid phase or solution phase libraries, synthetic libraries obtained by deconvolution or affinity chromatography selection, the “one-bead one-compound” libraries, and antibody libraries. See, e.g., Zuckermann et al. (1994) J. Med. Chem. 37, 2678-85; Lam (1997) Anticancer Drug Des. 12, 145; Lam et al. (1991) Nature 354, 82; Houghten et al. (1991) Nature 354, 84; and Songyang et al. (1993) Cell 72, 767. Examples of methods for the synthesis of molecular libraries can be found in the art, for example, in: DeWitt et al. (1993) Proc. Natl. Acad. Sci. USA 90, 6909; Erb et al. (1994) Proc. Natl. Acad. Sci. USA 91, 11422; Zuckermann et al. (1994) J. Med. Chem. 37, 2678; Cho et al. (1993) Science 261, 1303; Carrell et al. (1994) Angew. Chem. Int. Ed. Engl. 33, 2059; Carell et al. (1994) Angew. Chem. Int. Ed. Engl. 33, 2061; and Gallop et al. (1994) J. Med. Chem. 37,1233. Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (U.S. Pat. No. 5,223,409), spores (U.S. Pat. No. 5,223,409), plasmids (Cull et al. (1992) Proc. Natl. Acad. Sci. USA 89, 1865-1869), or phages (Scott and Smith (1990) Science 249, 386-390; Devlin (1990) Science 249, 404-406; Cwirla et al. (1990) Proc. Natl. Acad. Sci. USA 87, 6378-6382; Felici (1991) J. Mol. Biol. 222, 301-310; and U.S. Pat. No. 5,223,409).

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Identification of PGR-2

To identify genes up-regulated during adipogenesis, mRNA differential display analysis was performed using mouse 3T3-L1 cells. To induce adipogenesis, 3T3-L1 cells were treated with 1 μM dexamethasone and allowed to grow for 10 days at 37° C. A 199-nucleotide fragment was isolated and found to be highly expressed in 3T3-L1 cells harvested on the 10^{th} day after induction. The sequence of this fragment was determined to be identical to a segment of two GenBank entries, i.e., AK021033 and AK020666.

The full-length cDNA sequence corresponding to the coding region of the gene was referred to mouse PGR-2. This sequence was isolated and cloned from 3T3-L1 adipocytes as follows. PGR-2 cDNA was PCR-amplified and ligated into a pGEM-T easy vector (Promega) by T4 DNA ligase (Promega). The sequences of forward and reverse primers for amplifying PGR-2 cDNA were 5′-CGG TAT AGC TTG GGA CGC TA-3′ (SEQ ID NO:3) and 5′-TGC ATG TTA AGA ATC TTT GTG G-3′ (SEQ ID NO:4), respectively. The resulting construct (pTE-PGR-2) was then sequenced by T7 and SP6 polymerases. The coding region of PGR-2 open reading frame was then subcloned to the expression vector pCMV-Tag2B (Stratagene). For constructing pFLAG-PGR-2, a PCR reaction was conducted to generate a HindIII-SalI fragment of PGR-2 using pTE-PGR-2 as a template and two oligonucleotides as primers, 5′-AAC TGA AGC TTC AAG TGA TGA TCA TA-3′ (SEQ ID NO:5) and 5′-AGC TCT CCC ATA TGG TCG ACC T-3′ (SEQ ID NO:6). The PCR product thus obtained was then introduced into the HindIII-SalI sites of pCMV-Tag2B, yielding a fused construct of pFLAG/PGR-2. Finally, the pGEX-PGR-2 construct was prepared by ligating the HindIII-XhoI fragment of pFLAG/PGR-2 into a pGEX-4T-3 vector restricted with SmaI and XhoI (Pharmacia).

The deduced amino acid sequence of mouse PGR-2, i.e., SEQ ID NO:1, is shown above. The mouse PGR-2 was found to be homologous to two proteins: (1) human ZADH1 (GenBank accession no.: NM152444) with ˜92% homology, and (2) PGR/LTB4DH or PGR-1 with ˜54% homology.

PGR-2 expression increased during adipogenesis in 3T3-L1 cells. The maximal expression was observed at day 6 after induction of adipogenesis. At this time point, lipid droplets were observed to accumulate extensively in the adipocytes. The tissue distribution of PGR-2 was determined. It was highly expressed in adipose tissue. The amount of PGR-2 mRNA in omental fat was significantly higher in both homozygous and heterozygous db/db mice than in wild type mice.

Mouse PGR-2 was recombinantly expressed in E. coli as a GST fusion protein following standard procedures. The recombinant PGR-2 protein thus obtained was used to determine substrate specificity and enzymatic kinetics.

Enzymatic activity was determined as follows: 5 μg of recombinant mouse or human prostaglandin-Δ¹³-reductase 2/zinc binding alcohol dehydrogenase 1 (PGR2/ZADH1) protein was incubated at 37° C. in 50 μl of a reaction buffer containing 0.1 M Tris-HCl (pH 7.4),0.5 mM NADPH, and 0.57 mM 15-keto PGE₂. The reaction solution was kept in the dark for 2 hours at 37° C. and then mixed with 40 μl of a color development buffer containing 790 μM indonitrotetraolium chloride, 60 μM phenazene methosulfate, and 1% Tween 20 to oxidize any unreacted NADPH. After 10 min in the dark, 140 μl of a color development reagent containing 50 mM potassium hydrogen phthalate, pH 3.0, and 1% Tween 20 was added. Absorbance at 490 nm was measured using an ELISA plate reader. A standard curve was generated using reaction buffers containing serially diluted amounts of NADPH. Note that a specific activity of at least 90 nmole/min/mg protein indicates that the polypeptide has 15-keto prostaglandin-Δ¹³-reductase 2 activity.

Substrate specificity of PGR-2 was determined using the just-described procedure, except that 15-keto PGE₂ was replaced with each of six prostaglandin substrates, each of three downstream metabolites, or leukotriene B4. The substrates were purchased from Cayman Chemical Company (Michigan, USA). 15-keto PGE₁, 15-keto PGF_{1α}, and 15-keto PGF_{2α}, reacted specifically with PGR-2. By contrast, no specific activity was detected from 6-keto PGF1_{α}, 13,14-dihydro-15-keto PGE₂, and leukotriene B4.

Kinetics studies indicated that PGR-2 catalyzed reduction of 15-keto PGE₂, 15-keto PGE₁, 15-keto PGF_{2α}, 15-keto PGF_{1α}, 15-keto fluprostenol isopropyl ester, and 15-keto fluprostenol. Unlike PGR/LTB4DH, PGR-2 used NADPH as a cofactor much more efficiently than NADH.

The protein expression level of PGR-2 was up-regulated during adipogenesis in 3T3-L1 cells. The maximal PGR-2 protein level was detected in fully differentiated adipocytes. PPAR-γ was induced markedly at an earlier stage of adipogenesis. Low PGR-2 expression was localized in the nuclei in pre-adipocytes. Higher PGR-2 expression was distributed in the cytoplasm of the differentiated adipocytes.

Also investigated was the effect of PGR-2 expression on modulating PPAR-γ transcription in human Hep3B cells, which expressed endogenous human PPAR-α and -γ. Over-expression of PGR-2 in Hep3B cells was found to suppress PPAR-mediated transcriptional activation. The transcriptional activation was also suppressed even after Hep3B cells were stimulated by a PPAR-γagonist, i.e., BRL49653. Similar results were obtained from 3T3-L1 cells.

### Prostaglandin

The effect of prostaglandin on PPAR-γ activity in adipocytes was investigated. After treatment with a medium that induces cell differentiation, 3T3-L1 cells were treated from day 2 to 4 during adipogenesis with 14 μM 15-keto PGE₂, 13,14-dihydro-15-keto PGE₂, 15-keto PGF_{2α}, 13,14-dihydro-15-keto PGF_{2α}, or 4.5 μM of BRL49653 (a PPAR-γ agonist). See Forman et al., Cell (1995) 83:803-812. At day 6, aggregates of lipid droplets were stained with oil-red O for observation. 15-keto PGE₂ effectively enhanced adipogenesis at a level similar to BRL49653. After being induced to differentiate for two days, the 3T3-L1 cells were transfected with a reporter gene. Both 15-keto PGE₂ and 15-keto PGF_{2α} enhanced endogenous PPARs activity significantly. By contrast, the corresponding downstream metabolites, i.e., 13,14-dihydro-15-keto PGE₂ and 13,14-dihydro-15-keto PGF_{2α}, failed to increase PPARs activity.

A luciferase reporter gene was transfected to 3T3-L1 cells together with the ligand-binding domain of PPAR-α, PPAR-γ or PPAR-α fused to a yeast GAL4 DNA-binding domain. 15-keto PGE₂ and 15-keto PGF_{2α} activated PPAR-γ and, to a lesser degree, PPAR-α.

Also examined was the ability of 15-keto PGE₂ to induce protein expression of adipogenesis-specific, PPAR-γ target genes, i.e., IRS-1and -2. Substantial amounts of PPAR-γ1 and PPAR-γ2 protein were detected in 3T3-L1 cells when they were treated with insulin and dexamethasone, but not methylisobutylxanthine (MIX) alone. Addition of 15-keto PGE₂ and MIX with insulin and dexamethasone significantly enhanced PPAR-γ1 and PPAR-γ2 expression. 15-keto PGE₂ and BRL49653 strongly induced expression of aP2, an adipocyte-specifc marker, even in the absence of MIX. In the presence of insulin and dexamethasone, BRL49653 treatment dramatically increased IRS-2 expression. 15-keto PGE₂ enhanced the expression to a level similar to MIX. Either insulin/dexamethasone or MIX induced IRS-1 expression. PPAR-γ ligands including 15-keto PGE₂ and BRL49653 did not increase the amount of IRS-1 protein.

### PGR-2 Inhibitors

Recombinant human PGR/LTB4DH and PGR2/ZADH1 proteins were expressed and their enzymatic activities examined. Similar to mouse PGR-2, both recombinant human enzymes had PGR-2 activity and catalyzed conversion of 15-keto prostaglandin into 13,14-dihydro-15-keto prostaglandins.

Compounds 1-117 were tested for their inhibitory effects on PGR-2 activity. Compounds 1-6, 10-16, 18-23, 41, 44-67, 111, and 115-117 were acquired from Inodofine Chemical Co. Inc. (NJ, USA); compounds 7-9, 17, 28, 32, 79-82, and 112-114 were acquired from Sigma-Aldrich (MO, USA); compound 25, 30, 31, 37, 77, and 78 were acquired from SPEC (Netherland) ; compound 26 was acquired from Maybridge (UK); compounds 27, 35, 36, 68-75, and 98-101 were acquired from Chembridge (CA, USA); compound 24, 33, and 34 were acquired from Labotest (Germany); compounds 29, 38, and 108-110 were acquired from Dr. Ta-Jung Lu's lab at National Chung-Hsing University (Taichung, Taiwan); compounds 39-44 were acquired from Acme Bioscience (CA, USA); compounds 76, 92, 94, and 102-107 were acquired from Vardda Biotech (Mumbai, India); compounds of 83-91, 96, 97, and 106 were acquired from RYSS Lab (CA, USA) and compounds 94 and 96 were acquired from Dr. Hsu-Shan Huang's Lab at National Defense Medical Center (Taipei, Taiwan).

The inhibition assay was performed following the procedure described above. PGR-2 inhibitors were added to the reaction mixtures. The concentration of the inhibitors was 50 μM or 100 μM. The mixtures were then incubated for 2 hours at 37° C. It was found that all of compounds 1-117 inhibited 15-keto prostaglandin-Δ¹³-reductase 2 activity. Unexpectedly, compounds 8, 13, 14, 18, 27-29, 32-34, 40-46, 63, and 76 inhibited 15-keto prostaglandin-Δ¹³-reductase 2 activity by more than 50%.

The effect of compound 28 on insulin sensitivity was examined as follows. 3T3-L1cells were induced to differentiate in the same manner as described above. A glucose transport assay was performed by measuring uptake of 2-deoxy-D-[3H]glucose as described by Fingar et al. See Fingar et al., Endocrinology 134:728-735. 1.67 μM of the compound or 45 nM of a PPARs agonist, i.e., Avandia, was added to the cells. 10 μM cytochalasin-B was used to measure background glucose uptake levels. 100 nM of insulin was used to stimulate glucose uptake. Insulin treatment alone in differentiated 3T3-L1 cells increased glucose uptake by 20 folds. In the presence of compound 27, glucose uptake was increased by 30 folds. Of note, the ability of this compound to enhance glucose uptake was comparable to that of Avandia.

The in vivo effect compound 45 on glucose metabolism was also investigated. Female diabetic mice were obtained from the Jackson Laboratory (C57BLKS/J-m+/+Lep^{db}, 12 to 13 weeks old). These mice were characterized as insulin-resistant and hyperglycemic. 25 mg/kg of 2,2′-dihydroxychalcone (n=8) or placebo (n=4, final concentration against body fluid: 0.5% DMSO/1% ethanol) was injected intraperitoneally to the mice twice a day for two days. The mice were then fasted from the 48^{th} hour through 52^{nd} hour after the first injection. Blood samples were collected from the retro-orbital sinus before the first injection, and every two hours after two days of treatment. Blood glucose levels were measured by an electrode-type blood glucose meter (HORIBA, AntSense II, Japan). The compound treatment induced a significant reduction of blood glucose levels, compared to the placebo controls.

An RNA interference (RNAi) approach was used to silence PGR-2 expression. Two small interfering RNA (siRNA) duplexes, i.e., gaguucaguuuaccggaug (SEQ ID NO:7) and guucaagugaggacucuuu (SEQ ID NO:8), were annealed first and then introduced into 3T3-L1 fibroblasts or differentiating pre-adipocytes by transfection using oligofectamine (Invitrogen). Transfection of the siRNA duplexes reduced PGR-2 expression. In another experiment, transfection of the siRNA duplexes increased transcriptional activation of PPAR-γ. Thus, one can modulate PPAR-γ activity via silencing PGR-2 expression by RNA interference.

15-keto Prostaglandin Substrates 6p 5 μg of recombinant mouse or human prostaglandin-Δ¹³-reductase 2/zinc binding alcohol dehydrogenase 1 (PGR2/ZADH1) protein was incubated in 50 μl of a reaction buffer containing 0.1 M Tris-HCl (pH 7.4), 0.5 mM NADPH, and 0.57 mM a substrate. The substrate was 15-keto prostaglandin E1, 15-keto prostaglandin E2, 15-keto prostaglandin F1α, 15-keto prostaglandin F2α, 15-keto-fluprostenol isopropyl ester, or 15-keto-fluprostenol, which were purchased from Cayman Chemical Company (Michigan, USA). The reaction solution was kept for 2 hours at 37° C., and 20 μl of the reaction solution was mixed with 40 μl of a color development reagent containing 790 μM indonitrotetrazolium chloride, 60 μM phenazene methosulfate, and 1% Tween 20 to oxidize any unreacted NADPH. After 10-min reaction in the dark, 140 μl of a solution containing 50 mM potassium hydrogen phthalate (pH 3.0) and 1% Tween 20 was added. Absorbance at 490 nm was measured by an ELISA plate reader. A standard curve was generated using reaction buffers containing serially diluted amounts of NADPH.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of inhibiting 15-keto prostaglandin-Δ¹⁴-reductase 2, comprising contacting the 15-keto prostaglandin-Δ¹³-reductase 2 with an effective amount of a compound of formula (I): 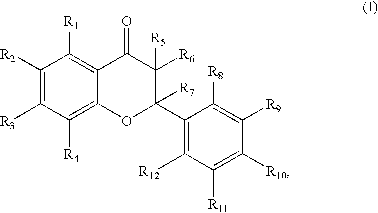
wherein
each of R₁, R₂, R₃, R₄, R₅, R₆, R₇, R₈, R₉, R₁₀, R₁₁, and R₁₂, independently, is H, OR, C₁₋₁₀ alkyl, C₃-C₂₀ cycloalkyl, C₃-C₂₀ heterocycloalkyl, aryl, or heteroaryl; in which R is H, C₁C₁₀ alkyl, C₃-C₂₀ cycloalkyl, C₃-C₂₀ heterocycloalkyl, aryl, or heteroaryl; or R₆ and R₇, taken together, represent a bond.

2. The method of claim 1, wherein each of R₁, R₂, R₃, R₄, R₅, R₈, R₉, R₁₀, R₁₁, and R₁₂, independently, is H or OR′, R′ being H, Me, or glucosyl.

3. The method of claim 2, wherein R₆ and R₇, taken together, represent a bond.

4. The method of claim 3, wherein each of R₅ is OH.

5. The method of claim 4, wherein each of R₁ and R₃ is H and R₂ is OH.

6. The method of claim 1, wherein the compound is:

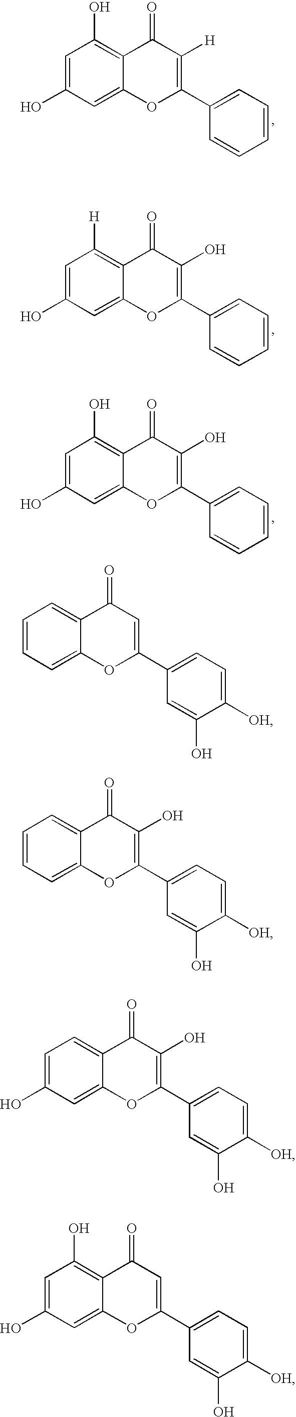

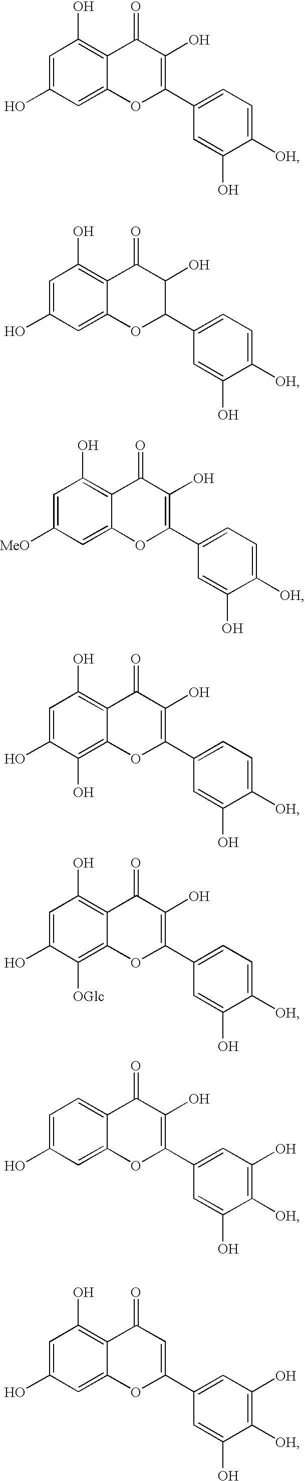

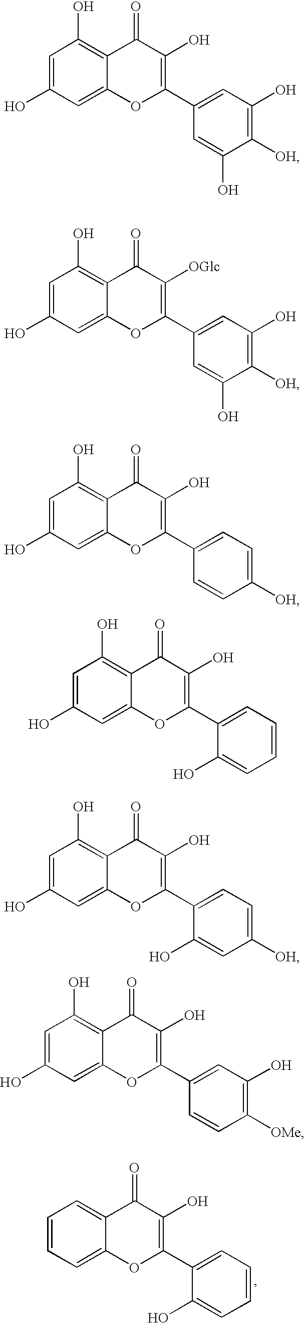

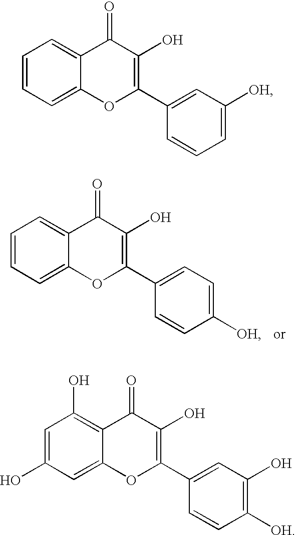

7. A method of inhibiting 15-keto prostaglandin-Δ¹³-reductase 2, comprising contacting the 15-keto prostaglandin-Δ¹³-reductase 2 with an effective amount of a compound of formula (II): 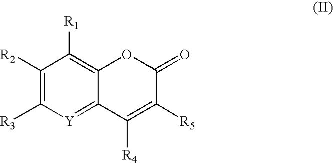
wherein:
Y is N or CR₆;
each of R₁, R₂, R₃, and R₆, independently, is H, halo, OR, C₁-C₁₀ alkyl, carboxy, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₁ and R₂, R₂ and R₃, or R₃ and R₆, together with the two carbon atoms to which they are attached, form C₃-C₂₀ cycloalkyl, C₃-C₂₀ heterocycloalkyl, aryl, or heteroaryl;
R₄ is H, halo, OR, C₁-C₁₀ alkyl, carboxy, C₃-C₂₀ cycloalkyl, C₃-C₂₀ heterocycloalkyl, aryl, or heteroaryl, in which R is defined above; and
R₅ is H, halo, OR, C₁-C₁₀ alkyl, carboxy, C₃-C₂₀ cycloalkyl, C₃-C₂₀ heterocycloalkyl, aryl, or heteroaryl, in which R is defined above; or R₅ is 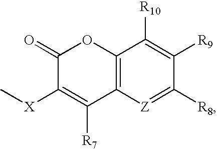
in which:
X is O, S, NR′, C(O), or CR′R″; each R′ and R″, independently, being H, OH, C₁-C₁₀ alkoxyl, halo, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R′ and R″, together with the carbon atom to which they are attached, being C₃-C₂₀ cycloalkyl, C₃-C₂₀ heterocycloalkyl, aryl, or heteroaryl;
Z is N or CR₁₁;
R₇ is H, OH, C₁-C₁₀ alkoxyl, halo, C₁-C₁₀ alkyl, C₃-C₂₀ cycloalkyl, C₃-C₂₀ heterocycloalkyl, aryl, or heteroaryl;
each of R₈, R₉, R₁₀, and R₁₁, independently, being H, OH, C₁-C₁₀ alkoxyl, halo, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₈ and R₉, R₉ and R₁₀, or R₈ and R₁₁, together with the two carbon atoms to which they are attached, form C₃-C₂₀ cycloalkyl, C₃-C₂₀ heterocycloalkyl, aryl, or heteroaryl.

8. The method of claim 7, wherein R₅ is

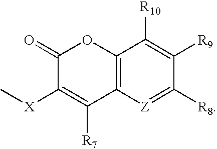

9. The method of claim 8, wherein Y is CR₆, Z is CR₁₁, and each of R₁, R₂, R₃, R₆, R₈, R₉, R₁₀, and R₁₁, independently, is H, OR, halo, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl.

10. The method of claim 9, wherein X is C(O) or CHR′, R′ being H, aryl or heteroaryl.

11. The method of claim 10, wherein each of R₁, R₂, R₃, R₄, R₅, R₇, R₈, R₉, R₁₀, and R₁₁, is H, OH, OMe, or halo.

12. The method of claim 7, wherein Y is CR₆ and each of R₁, R₂, R₃, R₄, R₅, and R₆, independently, is H, OR, C₁-C₁₀ alkyl, carboxy, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl.

13. The method of claim 12, wherein each of R₁, R₂, R₃, R₄, and R₆ is H, OH, OMe, or Me.

14. The method of claim 13, wherein R₅ is H or alkyl optionally substituted with carboxy, carbonyl, alkyloxycarbonyl, aryloxycarbonyl, or heteroaryl.

15. The method of claim 7, wherein the compound is

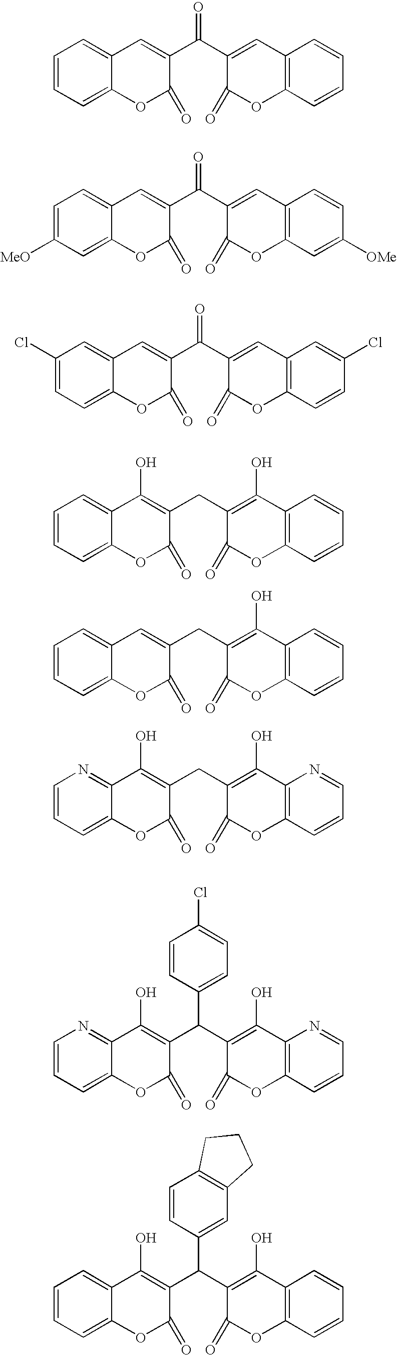

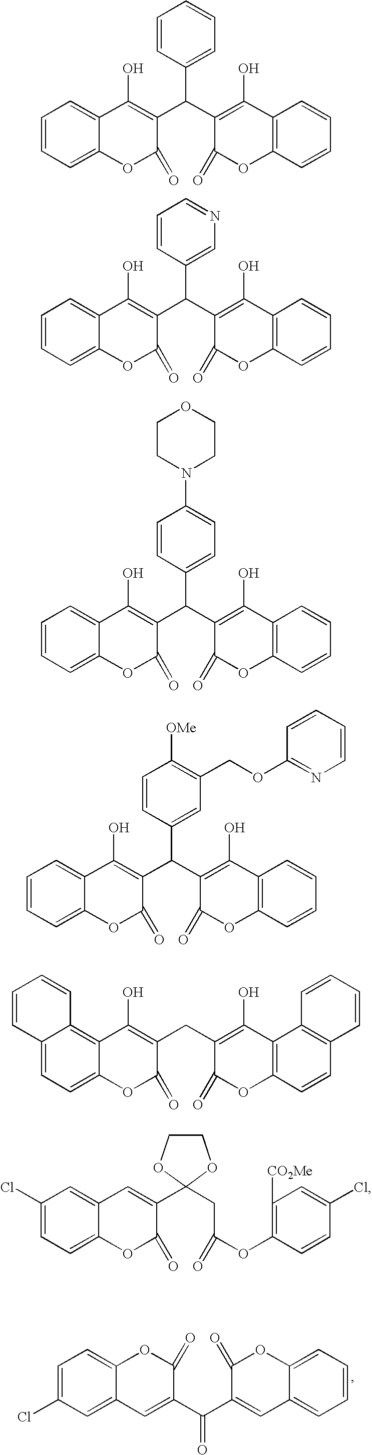

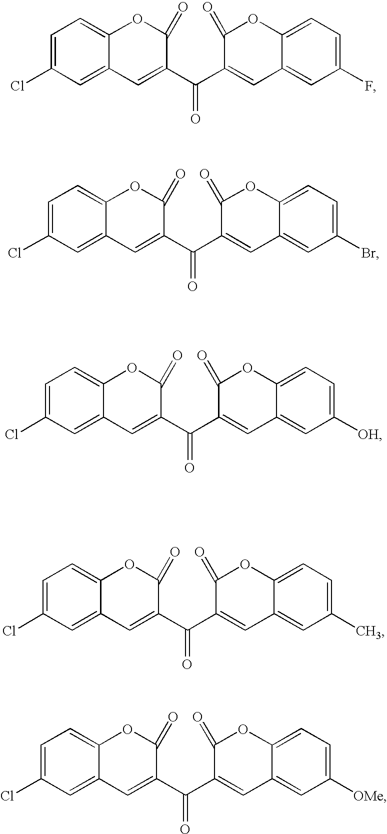

16. A method of inhibiting 15-keto prostaglandin-Δ¹³-reductase 2, comprising contacting the 15-keto prostaglandin-Δ¹³-reductase 2 with an effective amount of a compound of formula (III): 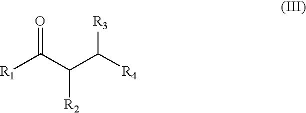
wherein
each of R₁ and R₄, independently, is H, OR, SR, NRR′, C₁-C₁₀ alkyl, C₃-C₂₀ cycloalkyl, C₃-C₂₀ heterocycloalkyl, aryl, or heteroaryl, in which each of R and R′, independently, is H, C₁-C₁₀ alkyl, C₃-C₂₀ cycloalkyl, C₃-C₂₀ heterocycloalkyl, aryl, or heteroaryl; and
each of R₂ and R₃, independently, is H, OR, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl; or R₂ and R₃, taken together, represent a single bond or double bond.

17. The method of claim 16, wherein each of R₁ and R₄, independently, is aryl or heteroaryl.

18. The method of claim 17, wherein R₁ is phenyl, optionally substituted with H, OR, C₁-C₁₀ alkyl, C₃-C₂₀ cycloalkyl, C₃-C₂₀ heterocycloalkyl, aryl, or heteroaryl, in which R is H, C₁-C₁₀ alkyl, C₃-C₂₀ cycloalkyl, C₃-C₂₀ heterocycloalkyl, aryl, or heteroaryl.

19. The method of claim 18, wherein R₂ and R₃, taken together, represent a single bond.

20. The method of claim 19, wherein R₄ is phenyl optionally substituted with OH, alkoxy, halo, nitro, cyano, alkyl, aryl, heterocylyl, or heteroaryl.

21. The method of claim 17, wherein each of R₆ and R₇ is H.

22. The method of claim 21, wherein R4 is furyl.

23. The method of claim 16, wherein the compound is:

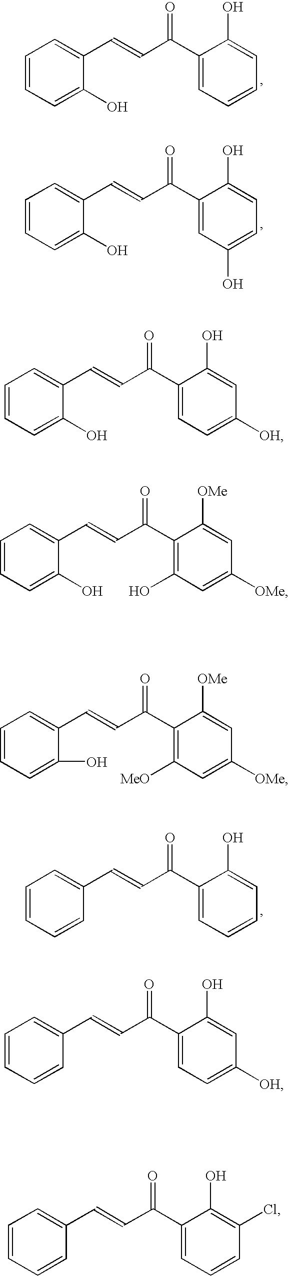

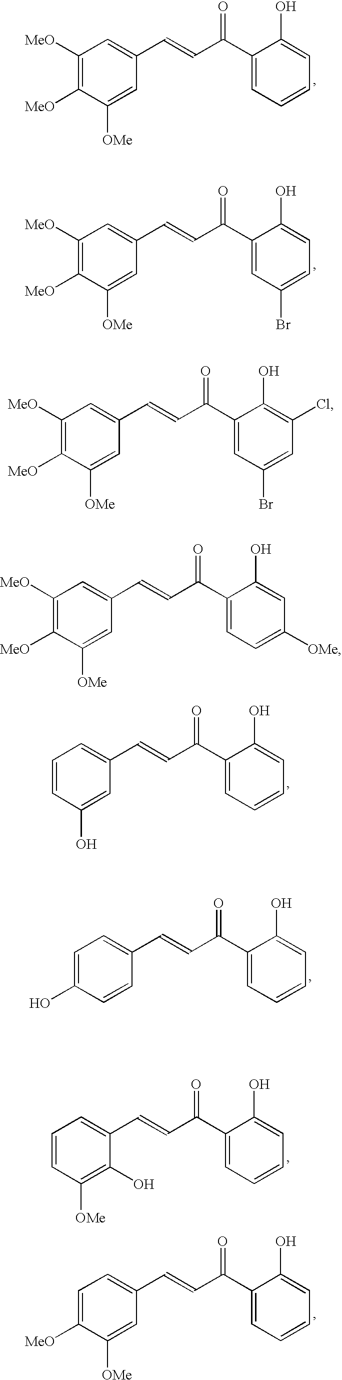

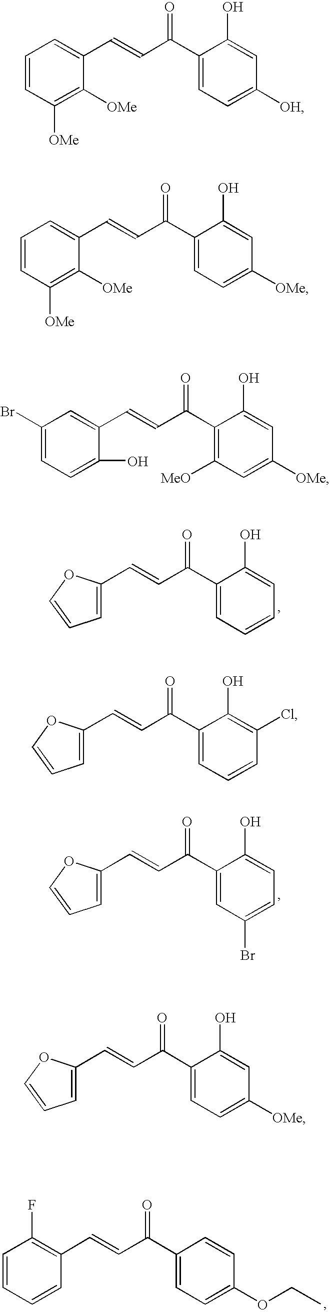

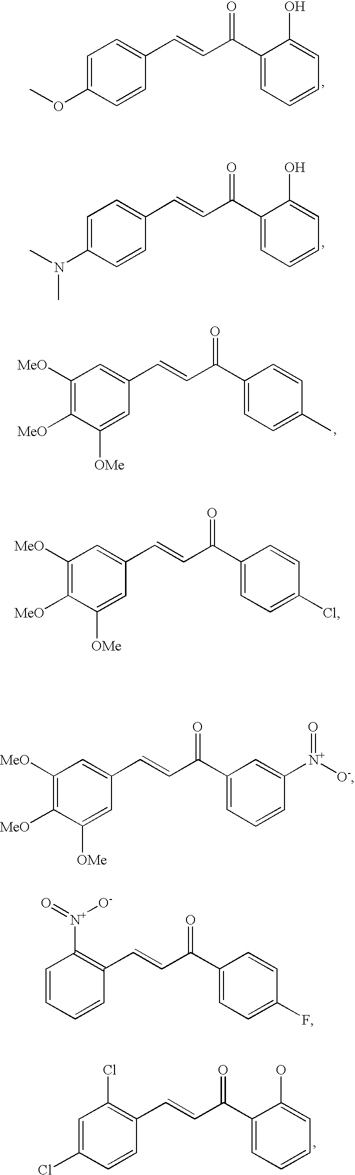

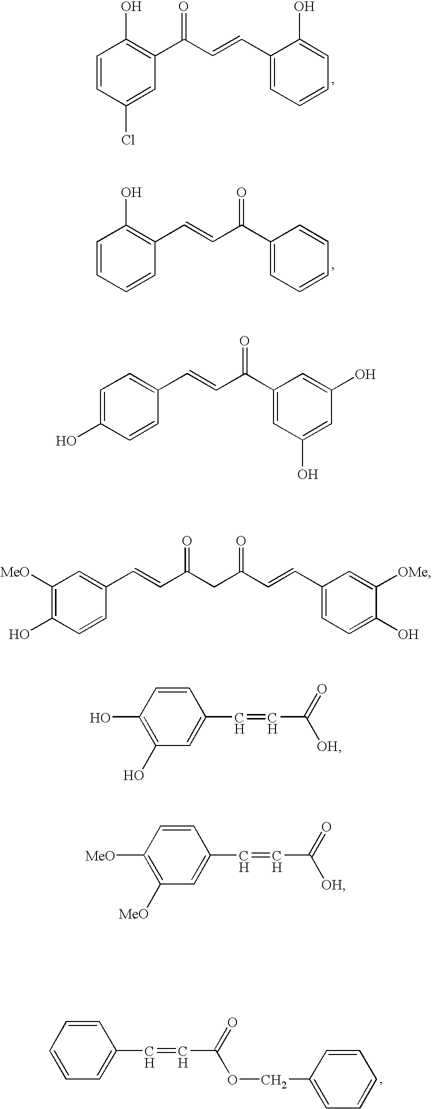

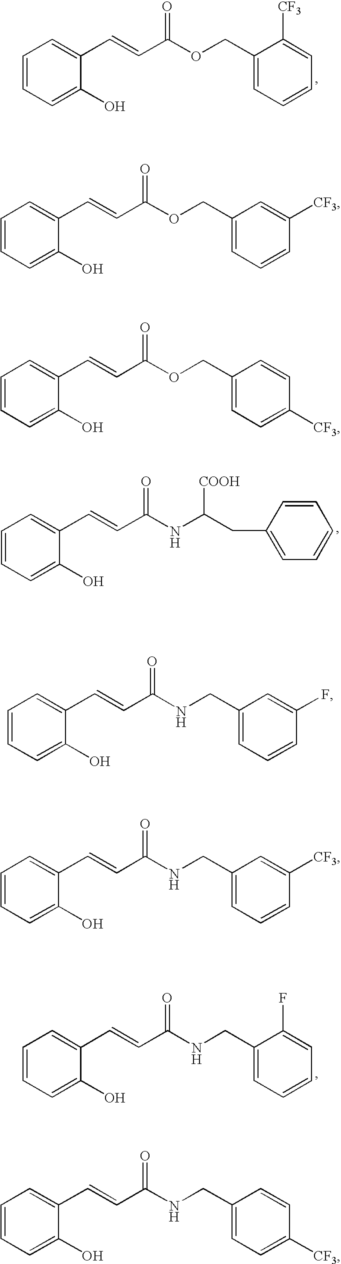

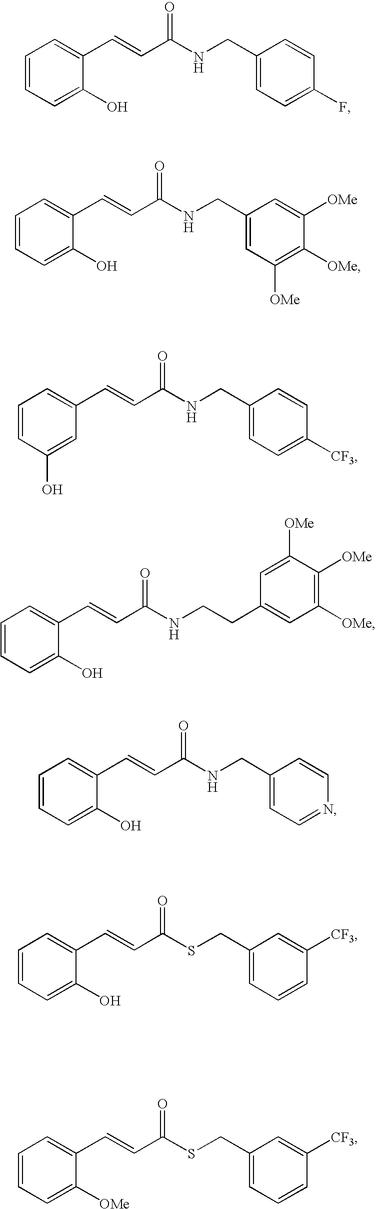

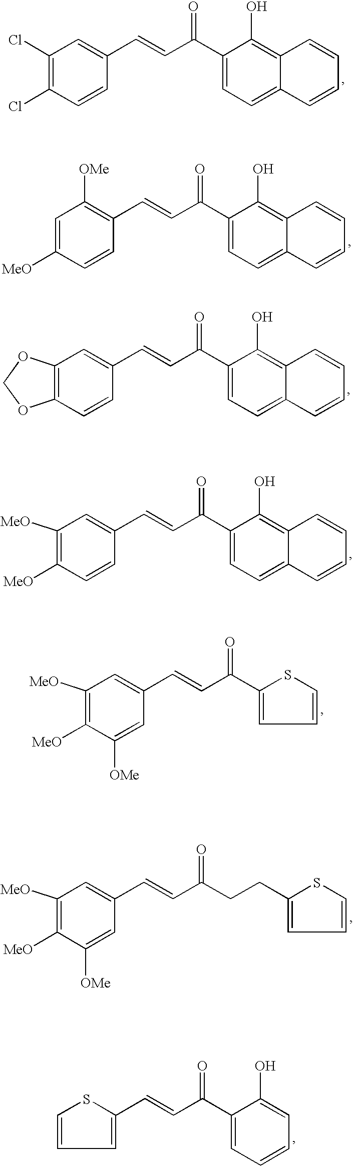

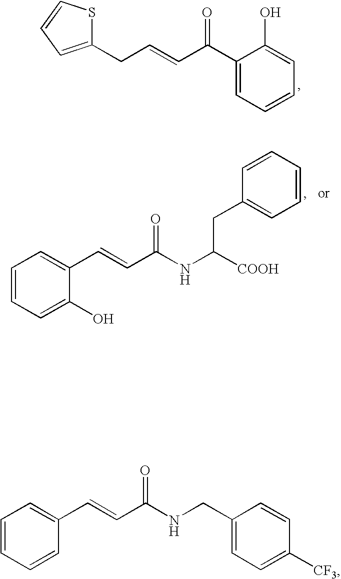

24. A method of inhibiting 15-keto prostaglandin-Δ¹³-reductase 2, comprising contacting the 15-keto prostaglandin-Δ¹³-reductase 2 with an effective amount of a compound of formula (IV): 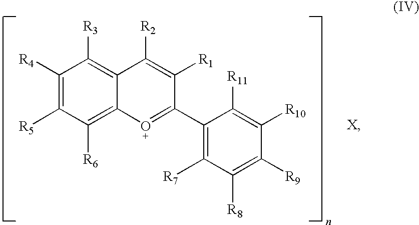
wherein
each of R₁, R₂, R₃, R₄, R₅, R₆, R₇, R₈, R₉, R₁₀, and R₁₁, independently, is H, OH, C₁-C₁₀ alkoxy, halo, C₁-C₁₀ alkyl, C₃-C₂₀ cycloalkyl, C₃-C₂₀ heterocycloalkyl, aryl, or heteroaryl; in which R is H, C₁-C₁₀ alkyl, C₃-C₂₀ cycloalkyl, C₃-C₂₀ heterocycloalkyl, aryl, or heteroaryl;
X is an anion; and
n is the absolute value of the charge of X.

25. The method of claim 24, wherein R₁, R₂, R₃, R₄, R₅, R₆, R₇, R₈, R₉, R₁₀, and R₁₁, independently, is H or OH.

26. The method of claim 24, where in the compound is:

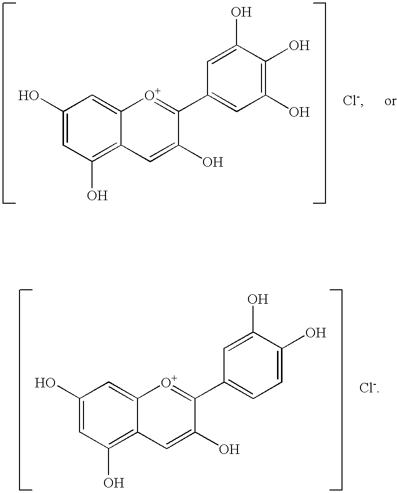

27. A method of treating a peroxisome proliferator-activated receptor (PPAR) related disease, comprising administering to a subject in need thereof an effective amount of a modulator of 15-keto prostaglandin-Δ¹³-reductase 2.

28. The method of claim 27, wherein the PPAR related disease is type II diabetes, obesity, dyslipidemia, coronary heart disease, inflammatory disease, or cancer.

29. The method of claim 28, wherein the PPAR related disease is type II diabetes.

30. The method of claim 29, wherein the modulator is 15-keto prostaglandin.

31. The method of claim 30, wherein the 15-keto prostaglandin is 15-keto PGE₂, 15-keto PGE1, 15-keto PGF2α, 15-keto PGF1α, 15-keto fluprostenol isopropyl ester, or 15-keto fluprostenol.

32. The method of claim 28, wherein the modulator is a compound of formula (I), (II), (III), or (IV).

33. A method of lowering blood glucose levels in a subject, comprising administering to a subject in need thereof an effective amount of a modulator of 15-keto prostaglandin-Δ¹³-reductase 2.

34. The method of claim 33, wherein the modulator is 15-keto prostaglandin.

35. The method of claim 34, wherein the 15-keto prostaglandin is 15-keto PGE₂, 15-keto PGE1, 15-keto PGF2α, 15-keto PGF1α, 15-keto fluprostenol isopropyl ester, or 15-keto fluprostenol.

36. The method of claim 33, wherein the modulator is a compound of formula (I), (II), (III), or (IV).
